(19) United States
(12) Patent Application Publication
(10) Pub. No.: US 2005/0158391 A1
(43) Pub. Date: Jul. 21, 2005

(54) Azithromycin multiparticulate dosage forms by melt-congeal processes

(75) Inventors: Leah E. Appel, Bend, OR (US); Roderick J. Ray, Bend, OR (US); David D. Newbold, Bend, OR (US); Dwayne T. Freisen, Bend, OR (US); Scott B. McCray, Bend, OR (US); James B. West, Bend, OR (US); David K. Lyon, Bend, OR (US); Marshall D. Crew, Bend, OR (US); Steven R. Lemott, East Lyme, CT (US); Scott M. Herbig, East Lyme, CT (US); Julian B. Lo, Old Lyme, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 11/003,856

(22) Filed: Dec. 3, 2004

Related U.S. Application Data
(60) Provisional application No. 60/527,244, filed on Dec. 4, 2003.

(51) Int. Cl. A61K 9/52; A61K 9/14; B29B 9/00; A61K 31/7052

(52) U.S. Cl. 424/489; 264/5; 514/28

(57) ABSTRACT

Azithromycin multiparticulates containing acceptably low concentrations of azithromycin esters are formed by a melt-congeal process.

### BACKGROUND OF THE INVENTION

Multiparticulates are well-known dosage forms that comprise a multiplicity of particles whose totality represents the intended therapeutically useful dose of a drug. When taken orally, multiparticulates generally disperse freely in the gastrointestinal tract, exit relatively rapidly and reproducibly from the stomach, maximize absorption, and minimize side effects. See, for example, Multiparticulate Oral Drug Delivery (Marcel Dekker, 1994), and Pharmaceutical Pelletization Technology (Marcel Dekker, 1989).

The preparation of drug particles by melting the drug, forming it into droplets and cooling the droplets to form small drug particles is known. Such processes for preparing multiparticulates are generally referred to as “melt-congeal” processes. See U.S. Pat. Nos. 4,086,346 and 4,092,089, both of which disclose rapid melting of phenacetin in an extruder and spraying the melt to form phenacetin granules.

Azithromycin is the generic name for the drug 9a-aza-9a-methyl-9-deoxo-9a-homoerythromycin A, a broad-spectrum antimicrobial compound derived from erythromycin A. Accordingly, azithromycin and certain derivatives thereof are useful as antibiotics.

It is well known that oral dosing of azithromycin can result in the occurrence of adverse side effects such as cramping, diarrhea, nausea and vomiting. Such side effects are higher at higher doses than at lower doses. Multiparticulates are a known improved dosage form of azithromycin that permit higher oral dosing with relatively reduced side effects. See U.S. Pat. No. 6,068,859. Such multiparticulates of azithromycin are particularly suitable for administration of single doses of the drug inasmuch as a relatively large amount of the drug can be delivered at a controlled rate over a relatively long period of time. A number of methods of formulating such azithromycin multiparticulates are disclosed in the '859 patent, including extrusion/spheronization, spray-drying, and spray-coating. However, often such processes and the inclusion of certain excipients in such multiparticulates can lead to degradation of the azithromycin during and after the process of forming the multiparticulates. The degradation occurs by virtue of a chemical reaction of the azithromycin with the components of the carriers or excipients used in forming the multiparticulates, resulting in the formation of azithromycin esters, a form of degradation of the azithromycin.

Published U.S. Application No. 2001/0006650A1 discloses the formation of “solid solution” beadlets by a spray-congealing method, the beadlets consisting of drug dissolved in a hydrophobic long chain fatty acid or ester, and a surfactant. However, azithromycin is not disclosed as a suitable drug for inclusion in the beadlets, there is no recognition in the disclosure of the problem of azithromycin ester formation, and there is no disclosure of the use of an extruder as an especially effective method of preparing a melt of the drug, the hydrophobic material and the surfactant.

The '859 patent also discloses the preparation of azithromycin-containing multiparticulates by stirring azithromycin with liquid wax to form a homogeneous mixture, cooling the mixture to a solid, then forcing the solid mixture through a screen to form granules. There are several drawbacks to such a process, including the possibility of azithromycin crystals being present on the surface of the multiparticulate, thereby exposing them to other azithromycin ester-forming excipients in a dosage form; the formation of non-uniformly sized and larger particles, leading to a larger particle size distribution; non-uniformity of azithromycin content owing to the settling of suspended drug during the time required to solidify the mixture; drug degradation caused by longer exposure to the liquid wax at elevated temperatures; non-uniformly shaped particles; and the risk of agglomeration of the particles.

What is therefore desired is a melt-congeal process for the formation of azithromycin multiparticulates wherein the aforementioned drawbacks are overcome and wherein excipients and process conditions are chosen to reduce the formation of azithromycin esters, resulting in a much greater degree of purity of the drug in multiparticulate dosage forms.

### BRIEF SUMMARY OF THE INVENTION

The present invention overcomes the drawbacks of the prior art by providing a melt-congeal process for forming multiparticulates comprising azithromycin and a pharmaceutically acceptable carrier that results in multiparticulates with acceptable concentrations of undesirable azithromycin esters.

According to the present invention it has been found that azithromycin ester formation is significantly suppressed in a number of ways: (1) by selection of a carrier from a particular class of materials which exhibit very low rates of ester formation with the drug; (2) by selection of processing parameters when a carrier is selected that has inherently higher rates of ester formation; and (3) by ensuring that the molten mixture of drug and carrier is of substantially uniform composition, preferably a homogeneous suspension of drug in the molten carrier, and that the residence time of the mixture in the melting means is minimized. A particularly effective means of accomplishing (3) is by the use of an extruder. It should be noted the drug and carrier mixture is “molten” in that a sufficient fraction of the mixture melts sufficiently that the material can be atomized to form droplets that can subsequently be congealed to form multiparticulates. However, typically much of the azithromycin and optionally a portion of the carrier may remain in the solid state. In the case of azithromycin, it is often preferable for as much as possible of the azithromycin to remain in the crystalline state. Thus, the “molten” mixture is often a suspension of solid drug and optionally excipients in molten carrier and drug.

An acceptable level of azithromycin ester formation is one which, during the time period beginning with formation of multiparticulates and continuing up until dosage, results in the formation of less than about 10 wt % azithromycin esters, meaning the weight of azithromycin esters relative to the total weight of azithromycin originally present in the multiparticulates, preferably less than about 5 wt %, more preferably less than about 1 wt %, even more preferably less than about 0.5 wt % and most preferably less than about 0.1 wt %.

Generically speaking, the class of carriers having inherently low rates of ester formation with azithromycin may be described as pharmaceutically acceptable carriers that contain no or relatively few acid and/or ester substituents as chemical substituents. All references to “acid and/or ester substituents” herein are to (1) carboxylic acid, sulfonic acid, and phosphoric acid substituents or (2) carboxylic acid ester, sulfonyl ester, and phosphate ester substituents, respectively. Conversely the class of carriers having inherently higher rates of ester formation with azithromycin may be described as pharmaceutically acceptable carriers that contain a relatively greater number of acid and/or ester substituents; within limits, processing conditions for this class of carriers may be utilized to suppress the rate of ester formation to an acceptable level.

Thus, in one aspect, the invention provides a process for forming multiparticulates comprising the steps (a) forming in an extruder a molten mixture comprising azithromycin and a pharmaceutically acceptable carrier, (b) delivering the molten mixture of step (a) to an atomizing means to form droplets from the molten mixture, and (c) congealing the droplets from step (b) to form multiparticulates.

In another aspect, the invention provides a process for forming multiparticulates comprises the steps (a) forming a molten mixture comprising azithromycin and a pharmaceutically acceptable carrier, (b) delivering the molten mixture of step (a) to an atomizing means to form droplets from the molten mixture, and (c) congealing the droplets from step (b) to form multiparticulates, wherein the concentration of azithromycin esters in the multiparticulates is less than about 10 wt %.

In both of the foregoing aspects, the processes of the present invention overcome the drawbacks of the above known methods used to form azithromycin multiparticulates.

One advantage of the processes of the present invention relative to known methods is that forming a molten mixture allows the carrier to wet the entire surface of the azithromycin drug crystals, thus allowing the drug crystals to be fully encapsulated by the carrier in the multiparticulate. Such encapsulation allows better control of the release of azithromycin from the multiparticulates and eliminates contact of the drug with other excipients in the dosage form.

Another advantage of the processes of the present invention relative to known methods is that they result in narrower particle size distributions relative to multiparticulates formed by mechanical means. Using atomization to form the droplets exploits the use of natural phenomenon such as surface tension to form spherical multiparticulates of uniform size. Particle size can be controlled through the atomization means, such as by adjusting the speed of a rotary atomizer.

Another advantage of the processes of the present invention relative to known methods is that they result in better content uniformity in that azithromycin containing droplets are formed that have relatively uniform drug content.

Still another advantage of the processes of the present invention relative to known methods is that they can reduce the amount of time the drug is in the molten state. The congealing step may occur rapidly, since the small droplets have a high surface area relative to volume.

Yet another advantage of the processes of the present invention relative to known methods is that they may be used to form smaller multiparticulates having a mean particle diameter as low as about 40 μm. Smaller particle size often results in better “mouth feel” for the patient.

In addition, the processes of the invention reduce the risk of multiparticulates agglomerating to one another. The atomization step often results in droplets that travel apart from one another during formation, allowing the multiparticulates to be formed separately from one another.

Finally, the processes of the present invention typically result in smoother, rounder particles relative to multiparticulates formed by mechanical means. This results in better flow characteristics that in turn facilitate processing.

### DETAILED DESCRIPTION OF PREFERRED EMBODIMENTS

As used in the present invention, the term “about” means the specified value ±10% of the specified value.

The compositions formed by the process of the present invention comprise a plurality of “multiparticulates.” The term “multiparticulate” is intended to embrace a dosage form comprising a multiplicity of particles whose totality represents the intended therapeutically useful dose of azithromycin. The particles generally are of a mean diameter from about 40 to about 3000 μm, preferably from about 50 to about 1000 μm, and most preferably from about 100 to about 300 μm. Multiparticulates are preferred because they are amenable to use in scaling dosage forms according to the weight of an individual patient in need of treatment by simply scaling the mass of particles in the dosage form to comport with the patient's weight. They are further advantageous since they allow the incorporation of a large quantity of drug into a simple dosage form such as a sachet that can be formulated into a slurry that can easily be consumed orally. Multiparticulates also have numerous therapeutic advantages over other dosage forms, especially when taken orally, including (1) improved dispersal in the gastrointestinal (GI) tract, (2) more uniform GI tract transit time, and (3) reduced inter- and intra-patient variability.

Azithromycin esters may be formed during the multiparticulate-forming process, during other processing steps required for manufacture of the finished dosage form, or during storage following manufacture but prior to dosing. Since the azithromycin dosage forms may be stored for up to two years or even longer prior to dosing, it is preferred that the concentration of azithromycin esters in the stored dosage form not exceed the above values prior to dosing.

While the multiparticulates can have any shape and texture, it is preferred that they be spherical, with a smooth surface texture. These physical characteristics lead to excellent flow properties, improved “mouth feel,” ease of swallowing and ease of uniform coating, if required.

The invention is particularly useful for administering relatively large amounts of azithromycin to a patient in a single-dose therapy. The amount of azithromycin contained within the multiparticulate dosage form is preferably at least 250 mgA, and can be as high as 7 gA (“mgA” and “gA” mean milligrams and grams of active azithromycin in the dosage form, respectively). The amount contained in the dosage form is preferably about 1.5 to about 4 gA, more preferably about 1.5 to about 3 gA, and most preferably 1.8 to 2.2 gA. For small patients, e.g., children weighing about 30 kg or less, the multiparticulate dosage form can be scaled according to the weight of the patient; in one aspect, the dosage form contains about 30 to about 90 mgA/kg of patient body weight, preferably about 45 to about 75 mgA/kg, more preferably, about 60 mgA/kg.

The multiparticulates formed by the process of the present invention are designed for controlled release of azithromycin after introduction to a use environment. As used herein, a “use environment” can be either the in vivo environment of the GI tract of a mammal, particularly a human, or the in vitro environment of a test solution. Exemplary test solutions include aqueous solutions at 37° C. comprising (1) 0.1 N HCl, simulating gastric fluid without enzymes; (2) 0.01 N HCl, simulating gastric fluid that avoids excessive acid degradation of azithromycin, and (3) 50 mM KH₂PO₄, adjusted to pH 6.8 using KOH, simulating intestinal fluid without enzymes. The inventors have also found that an in vitro test solution comprising 100 mM Na₂HPO₄, adjusted to pH 6.0 using NaOH provides a discriminating means to differentiate among different formulations on the basis of dissolution profile. It has been determined that in vitro dissolution tests in such solutions provide a good indicator of in vivo performance and bioavailability. Further details of in vitro tests and test solutions are described herein.

According to the present invention, reaction rates for excipients may be calculated so as to enable the practitioner to make an informed selection, following the general guideline that an excipient exhibiting a slower rate of ester formation is desirable, while an excipient exhibiting a faster rate of ester formation is undesirable.

### Melt-Congeal Process

The basic process used in the present invention comprises the steps of (a) forming a molten mixture comprising azithromycin and a pharmaceutically acceptable carrier, (b) delivering the molten mixture of step (a) to an atomizing means to form droplets from the molten mixture, and (c) congealing the droplets from step (b) to form multiparticulates.

The molten mixture comprises azithromycin and a pharmaceutically acceptable carrier. The azithromycin in the molten mixture may be dissolved in the carrier, may be a suspension of crystalline azithromycin distributed in the molten carrier, or any combination of such states or those states that are in between. Preferably the molten mixture is a homogeneous suspension of crystalline azithromycin in the molten carrier where the fraction of azithromycin that melts or dissolves in the molten carrier is kept relatively low. Preferably less than about 30 wt % of the total azithromycin melts or dissolves in the molten carrier. It is preferred that the azithromycin be present as the crystalline dihydrate.

Thus, “molten mixture” as used herein refers to a mixture of azithromycin and carrier heated sufficiently that the mixture becomes sufficiently fluid that the mixture may be formed into droplets or atomized. Atomization of the molten mixture may be carried out using any of the atomization methods described below. Generally, the mixture is molten in the sense that it will flow when subjected to one or more forces such as pressure, shear, and centrifugal force, such as that exerted by a centrifugal or spinning-disk atomizer. Thus, the azithromycin/carrier mixture may be considered “molten” when any portion of the mixture becomes sufficiently fluid that the mixture, as a whole, is sufficiently fluid that it may be atomized. Generally, a mixture is sufficiently fluid for atomization when the viscosity of the molten mixture is less than about 20,000 cp, preferably less than about 15,000 cp, and most preferably less than about 10,000 cp. Often, the mixture becomes molten when the mixture is heated above the melting point of one or more of the carrier components, in cases where the carrier is sufficiently crystalline to have a relatively sharp melting point; or, when the carrier components are amorphous, above the softening point of one or more of the carrier components. The molten mixture is therefore often a suspension of solid particles in a fluid matrix. In one preferred embodiment, the molten mixture comprises a mixture of substantially crystalline azithromycin particles suspended in a carrier that is substantially fluid. In such cases, a portion of the azithromycin may be dissolved in the fluid carrier and a portion of the carrier may remain solid.

Virtually any process may be used to form the molten mixture. One method involves heating the carrier in a tank until it is fluid and then adding the azithromycin to the molten carrier. Generally, the carrier is heated to a temperature of about 10° C. or more above the temperature at which it becomes fluid. The process is carried out so that at least a portion of the molten mixture remains fluid until atomized. Once the carrier has become fluid, the azithromycin may be added to the fluid carrier or “melt.” Although the term “melt” generally refers specifically to the transition of a crystalline material from its crystalline to its liquid state, which occurs at its melting point, and the term “molten” generally refers to such a crystalline material in its fluid state, as used herein, the terms are used more broadly, referring in the case of “melt” to the heating of any material or mixture of materials sufficiently that it becomes fluid in the sense that it may be pumped or atomized in a manner similar to a crystalline material in the fluid state. Likewise “molten” refers to any material or mixture of materials that is in such a fluid state. Alternatively, both the azithromycin and the solid carrier may be added to the tank and the mixture heated until the carrier has become fluid.

Once the carrier has become fluid and the azithromycin has been added, the mixture is mixed to ensure the azithromycin is substantially uniformly distributed therein. Mixing is generally done using mechanical means, such as overhead mixers, magnetically driven mixers and stir bars, planetary mixers, and homogenizers. Optionally, the contents of the tank can be pumped out of the tank and through an in-line, static mixer or extruder and then returned to the tank. The amount of shear used to mix the molten feed should be sufficiently high to ensure substantially uniform distribution of the azithromycin in the molten mixture. However, it is preferred that the shear not be so high such that the form of the azithromycin is changed, i.e., so as to cause a portion of the crystalline azithromycin to become amorphous or change to a new crystalline form of azithromycin. When the feed is a suspension of crystalline azithromycin in the carrier, it is also preferred that the shear not be so high as to substantially reduce the particle size of the azithromycin crystals. The feed solution can be mixed from a few minutes to several hours, the mixing time being dependent on the viscosity of the feed and the solubility of azithromycin in the carrier. Formation of esters can be further minimized by preventing dissolution of azithromycin up to its normal solubility limit by limiting the mixing time. Generally, it is preferred to limit the mixing time to near the minimum necessary to disperse the crystalline azithromycin substantially uniformly throughout the molten carrier.

When preparing the molten mixture using such a tank system in which the composition contains azithromycin in a crystalline hydrate or solvate form, the azithromycin can be maintained in this form by ensuring that the activity of water or solvent in the molten mixture is sufficiently high such that the waters of hydration or solvate of the azithromycin crystals are not removed by dissolution into the molten carrier. To keep the activity of water or solvent in the molten carrier high, it is desirable to keep the gas phase atmosphere above the molten mixture at a high water or solvent activity. The inventors have found that when crystalline azithromycin dihydrate is contacted with dry molten carrier and/or a dry gas-phase atmosphere, it can dissolve to a much greater extent into the molten carrier and also may be converted into other less stable amorphous or crystalline forms of azithromycin, such as the monohydrate. One method to ensure that crystalline azithromycin dihydrate is not converted to an amorphous crystalline form by virtue of loss of water of hydration is to humidify the head space in the mixing tank during the mixing. Alternatively, a small amount of water, on the order of 30 to 100 wt % of the solubility of water in the molten carrier at the process temperature can be added to the feed to ensure there is sufficient water to prevent loss of the azithromycin dihydrate crystalline form. Humidification of the headspace and addition of water to the feed may also be combined and good results obtained. This is disclosed more fully in commonly assigned U.S. Patent Application Ser. No. 60/527,316 (“Method for Making Pharmaceutical Multiparticulates,” Attorney Docket No. PC25021), filed Dec. 4, 2003.

An alternative method of preparing the molten mixture is to use two tanks, melting a first carrier in one tank and a second in another. The azithromycin is added to one of these tanks and mixed as described above. The same precautions regarding the activity of water in the tanks should be taken with such a dual tank system. The two melts are then pumped through an in-line static mixer or extruder to produce a single molten mixture that is directed to the atomization process described below. Such a dual system has advantages when one of the excipients has a high reactivity with azithromycin or when the excipients are mutually reactive, such as when one carrier is a crosslinking agent that reacts with the second carrier to form a crosslinked multiparticulate. An example of the latter is the use of an ionic crosslinking agent with alginic acid as the excipient.

Another method that can be used to prepare the molten mixture is to use a continuously stirred tank system. In this system, the azithromycin and carrier are continuously added to a heated tank equipped with means for continuous stirring, while the molten mixture is continuously removed from the tank. The contents of the tank are heated sufficiently that the temperature of the contents is about 10° C. or more above the temperature at which the molten mixture becomes fluid. The azithromycin and carrier are added in such proportions that the molten feed removed from the tank has the desired composition. The azithromycin is typically added in solid form and may be pre-heated prior to addition to the tank. If added in a hydrated crystalline form and preheated, the azithromycin should be heated under conditions with sufficiently high water activity, typically 30 to 100% RH, to prevent dehydration and consequent conversion of the azithromycin crystalline form as previously stated. The carrier may also be preheated or even pre-melted prior to addition to the continuously stirred tank system. A wide variety of mixing methods can be used with such a system, such as those described above.

The molten mixture may also be formed using a continuous mill, such as a Dyno® Mill wherein solid azithromycin and carrier are fed to the mill's grinding chamber containing grinding media, such as beads with diameters of 0.25 to 5 mm. The grinding chamber typically is jacketed so heating or cooling fluid may be circulated around the chamber to control the temperature in the chamber. The molten mixture is formed in the grinding chamber, and exits the chamber through a separator to remove the grinding media from the molten mixture.

An especially preferred method of forming the molten mixture is by an extruder. By “extruder” is meant a device or collection of devices that creates a molten extrudate by heat and/or shear forces and/or produces a uniformly mixed extrudate from a solid and/or liquid (e.g., molten) feed. Such devices include, but are not limited to single-screw extruders; twin-screw extruders, including co-rotating, counter-rotating, intermeshing, and non-intermeshing extruders; multiple screw extruders; ram extruders, consisting of a heated cylinder and a piston for extruding the molten feed; gear-pump extruders, consisting of a heated gear pump, generally counter-rotating, that simultaneously heats and pumps the molten feed; and conveyer extruders. Conveyer extruders comprise a conveyer means for transporting solid and/or powdered feeds, such, such as a screw conveyer or pneumatic conveyer, and a pump. At least a portion of the conveyer means is heated to a sufficiently high temperature to produce the molten mixture. The molten mixture may optionally be directed to an accumulation tank, before being directed to a pump, which directs the molten mixture to an atomizer. Optionally, an in-line mixer may be used before or after the pump to ensure the molten mixture is substantially homogeneous. In each of these extruders the molten mixture is mixed to form a uniformly mixed extrudate. Such mixing may be accomplished by various mechanical and processing means, including mixing elements, kneading elements, and shear mixing by backflow. Thus, in such devices, the composition is fed to the extruder, which produces a molten mixture that can be directed to the atomizer.

In one embodiment, the composition is fed to the extruder in the form of a solid powder. The powdered feed can be prepared using methods well known in the art for obtaining powdered mixtures with high content uniformity. See Remington's Pharmaceutical Sciences (16th ed. 1980). Generally, it is desirable that the particle sizes of the azithromycin and carrier be similar to obtain a uniform blend. However, this is not essential to the successful practice of the invention.

An example of a process for preparing the powdered feed is as follows: first, the carrier is milled so that its particle size is about the same as that of the azithromycin; next, the azithromycin and carrier are blended in a V-blender for 20 minutes; the resulting blend is then de-lumped to remove large particles and finally blended for an additional 4 minutes. In some cases it is difficult to mill the carrier to the desired particle size since many of these materials tend to be waxy substances and the heat generated during the milling process can gum up the milling equipment. In such cases, small particles of the carrier can be formed using a melt-congeal process, as described below. The resulting congealed particles of carrier can then be blended with the azithromycin to produce the feed for the extruder.

Another method for producing the powdered feed to the extruder is to melt the carrier in a tank, mix in the azithromycin as described above for the tank system, and then cool the molten mixture, producing a solidified mixture of azithromycin and carrier. This solidified mixture can then be milled to a uniform particle size and fed to the extruder.

A two-feed extruder system can also be used to produce the molten mixture. In this system the carrier and azithromycin, both in powdered form, are fed to the extruder through the same or different feed ports. In this way, the need for blending the components is eliminated.

Alternatively, the carrier in powder form may be fed to the extruder at one point, allowing the extruder to melt the carrier. The azithromycin is then added to the molten carrier through a second feed delivery port part way along the length of the extruder, thus reducing the contact time of the azithromycin with the molten carrier, thereby further reducing the formation of azithromycin esters. The closer the second feed delivery port is to the extruder's discharge port, the lower is the residence time of azithromycin in the extruder. Multiple-feed extruders can be used when the carrier comprises more than one excipient.

In another method, the composition is in the form of larger solid particles or a solid mass, rather than a powder, when fed to the extruder. For example, a solidified mixture can be prepared as described above and then molded to fit into the cylinder of a ram extruder and used directly without milling.

In another method, the carrier can be first melted in, for example, a tank, and fed to the extruder in molten form. The azithromycin, typically in powdered form, may then be introduced to the extruder through the same or a different delivery port used to feed the carrier into the extruder. This system has the advantage of separating the melting step for the carrier from the mixing step, reducing contact of the azithromycin with the molten carrier and further reducing the formation of azithromycin esters.

In each of the above methods, the extruder should be designed such that it produces a molten mixture, preferably with azithromycin crystals uniformly distributed in the carrier. Generally, the temperature of the extrudate should be about 10° C. or more above the temperature at which the azithromycin and carrier mixture becomes fluid. In cases where the carrier is a single crystalline material, this temperature is typically about 10° C. or more above the melting point of the carrier. The various zones in the extruder should be heated to appropriate temperatures to obtain the desired extrudate temperature as well as the desired degree of mixing or shear, using procedures well known in the art. As noted above for mechanical mixing, the shear level is preferably relatively low, yet sufficient to produce a substantially uniform molten mixture.

In cases where the carrier has a high reactivity with azithromycin, the residence time of material in the extruder should be kept as short as is practical in order to further limit the formation of azithromycin esters. In such cases the extruder should be designed so that time necessary to produce a molten mixture with the crystalline azithromycin uniformly distributed is sufficiently short that the formation of azithromycin esters is kept at an acceptable level. Methods for designing the extruder so as to achieve shorter residence times are known in the art. The residence time in the extruder should then be kept sufficiently low that azithromycin ester formation is kept at or below an acceptable level.

As described above for other methods of forming the molten feed mixture, when a crystalline hydrate, such as the dihydrate form of azithromycin is used, it will be desirable to maintain a high water activity in the drug/carrier admixture to reduce dehydration of the azithromycin. This can be accomplished either by adding water to the powdered feed blend or by injecting water directly into the extruder by metering a controlled amount of water into a separate delivery port. In either case, sufficient water should be added to ensure the water activity is high enough to maintain the desired form of the crystalline azithromycin. When the azithromycin is in the dihydrate crystalline form, it is desirable to keep the water activity of any material in contact with azithromycin in the 30% RH to 100% RH range. This can be accomplished by ensuring that the concentration of water in the molten carrier is 30% to 100% of the solubility of water in the molten carrier at the maximum process temperature. In some cases, a small excess of water above the 100% water solubility limit may be added to the mixture.

Once the molten mixture has been formed, it is delivered to an atomizer that breaks the molten feed into small droplets. Virtually any method can be used to deliver the molten mixture to the atomizer, including the use of pumps and various types of pneumatic devices such as pressurized vessels or piston pots. When an extruder is used to form the molten mixture, the extruder itself can be used to deliver the molten mixture to the atomizer. Typically, the molten mixture is maintained at an elevated temperature while delivering the mixture to the atomizer to prevent solidification of the mixture and to keep the molten mixture flowing.

Generally, atomization occurs in one of several ways, including (1) by “pressure” or single-fluid nozzles; (2) by two-fluid nozzles; (3) by centrifugal or spinning-disk atomizers, (4) by ultrasonic nozzles; and (5) by mechanical vibrating nozzles. Detailed descriptions of atomization processes can be found in Lefebvre, Atomization and Sprays (1989) or in Perry's Chemical Engineers' Handbook (7th Ed. 1997).

There are many types and designs of pressure nozzles, which generally deliver the molten mixture at high pressure to an orifice. The molten mixture exits the orifice as a filament or as a thin sheet that breaks up into filaments, which subsequently break up into droplets. The operating pressure drop across the pressure nozzle ranges from 1 barg to 70 barg, depending on the viscosity of the molten feed, the size of the orifice, and the desired size of the multiparticulates.

In two-fluid nozzles, the molten mixture is contacted with a stream of gas, typically air or nitrogen, flowing at a velocity sufficient to atomize the molten mixture. In internal-mixing configurations, the molten mixture and gas mix inside the nozzle before discharging through the nozzle orifice. In external-mixing configurations, high velocity gas outside the nozzle contacts the molten mixture. The pressure drop of gas across such two-fluid nozzles typically ranges from 0.5 barg to 10 barg.

In centrifugal atomizers, also known as rotary atomizers or spinning-disk atomizers, the molten mixture is fed onto a rotating surface, where it is caused to spread out by centrifugal force. The rotating surface may take several forms, examples of which include a flat disk, a cup, a vaned disk, and a slotted wheel. The surface of the disk may also be heated to aid in formation of the multiparticulates. Several mechanisms of atomization are observed with flat-disk and cup centrifugal atomizers, depending on the flow of molten mixture to the disk, the rotation speed of the disk, the diameter of the disk, the viscosity of the feed, and the surface tension and density of the feed. At low flow rates, the molten mixture spreads out across the surface of the disk and when it reaches the edge of the disk, forms a discrete droplet, which is then flung from the disk. As the flow of molten mixture to the disk increases, the mixture tends to leave the disk as a filament, rather than as a discrete droplet. The filament subsequently breaks up into droplets of fairly uniform size. At even higher flow rates, the molten mixture leaves the disk edge as a thin continuous sheet, which subsequently disintegrates into irregularly sized filaments and droplets. The diameter of the rotating surface generally ranges from 2 cm to 50 cm, and the rotation speeds range from 500 rpm to 100,000 rpm or higher, depending on the desired size of the multiparticulates.

In ultrasonic nozzles, the molten mixture is fed through or over a transducer and horn, which vibrates at ultrasonic frequencies, atomizing the molten mixture into small droplets. In mechanical vibrating nozzles, the molten mixture is fed through a needle vibrating at a controlled frequency, atomizing the molten mixture into small droplets. In both cases, the particle size produced is determined by the liquid flow rate, frequency of ultrasound or vibration, and the orifice diameter.

In a preferred embodiment, the atomizer is a centrifugal or spinning-disk atomizer, such as the FX1 100-mm rotary atomizer manufactured by Niro A/S (Soeborg, Denmark).

The molten mixture comprising azithromycin and a carrier is delivered to the atomization process as a molten mixture, as described above.

Preferably, the feed is molten prior to congealing for at least 5 seconds, more preferably at least 10 seconds, and most preferably at least 15 seconds so as to ensure adequate homogeneity of the drug/carrier melt. It is also preferred that the molten mixture remain molten for no more than about 20 minutes to limit formation of azithromycin esters. As described above, depending on the reactivity of the chosen carrier, it may be preferable to further reduce the time that the azithromycin mixture is molten to well below 20 minutes in order to further limit azithromycin ester formation to an acceptable level. In such cases, such mixtures may be maintained in the molten state for less than 15 minutes, and in some cases, even less than 10 minutes. When an extruder is used to produce the molten feed, the times above refer to the mean time from when material is introduced to the extruder to when the molten mixture is congealed. Such mean times can be determined by procedures well known in the art. For example, a small amount of dye or other tracer substance is added to the feed while the extruder is operating under nominal conditions. Congealed multiparticulates are then collected over time and analyzed for the dye or tracer substance, from which the mean time is determined. In a particularly preferred embodiment the azithromycin is maintained substantially in the crystalline dihydrate state. To accomplish this, the feed is preferably hydrated by addition of water to a relative humidity of at least 30% at the maximum temperature of the molten mixture.

Once the molten mixture has been atomized, the droplets are congealed, typically by contact with a gas or liquid at a temperature below the solidification temperature of the droplets. Typically, it is desirable that the droplets are congealed in less than about 60 seconds, preferably in less than about 10 seconds, more preferably in less than about 1 second. Often, congealing at ambient temperature results in sufficiently rapid solidification of the droplets to avoid excessive azithromycin ester formation. However, the congealing step often occurs in an enclosed space to simplify collection of the multiparticulates. In such cases, the temperature of the congealing media (either gas or liquid) will increase over time as the droplets are introduced into the enclosed space, leading to the possible formation of azithromycin esters. Thus, a cooling gas or liquid is often circulated through the enclosed space to maintain a constant congealing temperature. When the carrier used is highly reactive with azithromycin, the time the azithromycin is exposed to the molten carrier must be kept to an acceptably low level. In such cases, the cooling gas or liquid can be cooled to below ambient temperature to promote rapid congealing, thus further reducing the formation of azithromycin esters.

In preferred embodiments, the azithromycin in the multiparticulates is in the form of a crystalline hydrate, such as the crystalline dihydrate. To maintain the crystalline hydrate form and prevent conversion to other crystalline forms, the water concentration in the congealing atmosphere or liquid should be kept high to avoid loss of the waters of hydration, as previously noted. Generally, the humidity of the congealing medium should be maintained at 30% RH or higher to maintain the crystalline form of the azithromycin.

### Azithromycin

The multiparticulates of the present invention comprise azithromycin. Preferably, the azithromycin makes up from about 5 wt % to about 90 wt % of the total weight of the multiparticulate, more preferably from about 10 wt % to about 80 wt %, and even more preferably from about 30 wt % to about 60 wt % of the total weight of the multiparticulates.

As used herein, “azithromycin” means all amorphous and crystalline forms of azithromycin including all polymorphs, isomorphs, pseudomorphs, clathrates, salts, solvates and hydrates of azithromycin, as well as anhydrous azithromycin. Reference to azithromycin in terms of therapeutic amounts or in release rates in the claims is to active azithromycin, i.e., the non-salt, non-hydrated azalide molecule having a molecular weight of 749 g/mole.

Preferably, the azithromycin of the present invention is azithromycin dihydrate, which is disclosed in U.S. Pat. No. 6,268,489.

In alternate embodiments of the present invention, the azithromycin comprises a non-dihydrate azithromycin, a mixture of non-dihydrate azithromycins, or a mixture of azithromycin dihydrate and non-dihydrate azithromycins. Examples of suitable non-dihydrate azithromycins include, but are not limited to, alternate crystalline forms B, D, E, F, G, H, J, M, N, O, P, Q and R.

Azithromycin also occurs as Family I and Family II isomorphs, which are hydrates and/or solvates of azithromycin. The solvent molecules in the cavities have a tendency to exchange between solvent and water under specific conditions. Therefore, the solvent/water content of the isomorphs may vary to a certain extent.

Azithromycin form B, a hygroscopic hydrate of azithromycin, is disclosed in U.S. Pat. No. 4,474,768.

Azithromycin forms D, E, F, G, H, J, M, N, O, P, Q and R are disclosed in commonly owned U.S. Patent Publication No. 20030162730, published Aug. 28, 2003.

Forms B, F, G, H, J, M, N, O, and P belong to Family I azithromycin and have a monoclinic P2₁ space group with cell dimensions of a=16.3±0.3 Å, b=16.2±0.3 Å, c=18.4±0.3 Å and beta=109±2°.

Form F azithromycin is an azithromycin ethanol solvate of the formula C₃₈H₇₂N₂O₁₂.H₂O.0.5C₂H₅OH in the single crystal structure and is an azithromycin monohydrate hemi-ethanol solvate. Form F is further characterized as containing 2-5 wt % water and 1-4 wt % ethanol by weight in powder samples. The single crystal of form F is crystallized in a monoclinic space group, P2₁, with the asymmetric unit containing two azithromycin molecules, two water molecules, and one ethanol molecule, as a monohydrate/hemi-ethanolate. It is isomorphic to all Family I azithromycin crystalline forms. The theoretical water and ethanol contents are 2.3 and 2.9 wt %, respectively.

Form G azithromycin has the formula C₃₈H₇₂N₂O₁₂.1.5H₂O in the single crystal structure and is an azithromycin sesquihydrate. Form G is further characterized as containing 2.5-6 wt % water and <1 wt % organic solvent(s) by weight in powder samples. The single crystal structure of form G consists of two azithromycin molecules and three water molecules per asymmetric unit, corresponding to a sesquihydrate with a theoretical water content of 3.5 wt %. The water content of powder samples of form G ranges from about 2.5 to about 6 wt %. The total residual organic solvent is less than 1 wt % of the corresponding solvent used for crystallization.

Form H azithromycin has the formula C₃₈H₇₂N₂O₁₂.H₂O.0.5C₃H₈O₂ and may be characterized as an azithromycin monohydrate hemi-1,2 propanediol solvate. Form H is a monohydrate/hemi-propylene glycol solvate of azithromycin free base.

Form J azithromycin has the formula C₃₈H₇₂N₂O₁₂.H₂O.0.5C₃H₇OH in the single crystal structure, and is an azithromycin monohydrate hemi-n-propanol solvate. Form J is further characterized as containing 2-5 wt % water and 1-5 wt % n-propanol by weight in powder samples. The calculated solvent content is about 3.8 wt % n-propanol and about 2.3 wt % water.

Form M azithromycin has the formula C₃₈H₇₂N₂O₁₂.H₂O.0.5C₃H₇OH, and is an azithromycin monohydrate hemi-isopropanol solvate. Form M is further characterized as containing 2-5 wt % water and 1-4 wt % 2-propanol by weight in powder samples. The single crystal structure of form M would be a monohydrate/hemi-isopropranolate.

Form N azithromycin is a mixture of isomorphs of Family I. The mixture may contain variable percentages of isomorphs F, G, H, J, M and others, and variable amounts of water and organic solvents, such as ethanol, isopropanol, n-propanol, propylene glycol, acetone, acetonitrile, butanol, pentanol, etc. The weight percent of water can range from 1-5.3 wt % and the total weight percent of organic solvents can be 2-5 wt % with each solvent making up 0.5-4 wt %.

Form O azithromycin has the formula C₃₈H₇₂N₂O₁₂.0.5H₂O.0.5C₄H₉OH, and is a hemihydrate hemi-n-butanol solvate of azithromycin free base by single crystal structural data.

Form P azithromycin has the formula C₃₈H₇₂N₂O₁₂.H₂O.0.5C₅H₁₂O and is an azithromycin monohydrate hemi-n-pentanol solvate.

Form Q is distinct from Families I and II, has the formula C₃₈H₇₂N₂O₁₂.H₂O.0.5C₄H₈O and is an azithromycin monohydrate hemi-tetrahydrofuran (THF) solvate. It contains about 4% water and about 4.5 wt % THF.

Forms D, E and R belong to Family II azithromycin and contain an orthorhombic P2₁ 2₁2₁ space group with cell dimensions of a=8.9±0.4 Å, b=12.3±0.5 Å and c=45.8±0.5 Å.

Form D azithromycin has the formula C₃₈H₇₂N₂O₁₂.H₂O.C₆H₁₂ in its single crystal structure, and is an azithromycin monohydrate monocyclohexane solvate. Form D is further characterized as containing 2-6 wt % water and 3-12 wt % cyclohexane by weight in powder samples. From single crystal data, the calculated water and cyclohexane content of form D is 2.1 and 9.9 wt %, respectively.

Form E azithromycin has the formula C₃₈H₇₂N₂O₁₂.H₂O.C₄H₈O and is an azithromycin monohydrate mono-THF solvate by single crystal analysis.

Form R azithromycin has the formula C₃₈H₇₂N₂O₁₂.H₂O.C₅H₁₂O and is an azithromycin monohydrate mono-methyl tert-butyl ether solvate. Form R has a theoretical water content of 2.1 wt % and a theoretical methyl tert-butyl ether content of 10.3 wt %.

Other examples of non-dihydrate azithromycin include, but are not limited to, an ethanol solvate of azithromycin or an isopropanol solvate of azithromycin. Examples of such ethanol and isopropanol solvates of azithromycin are disclosed in U.S. Pat. Nos. 6,365,574 and 6,245,903 and U.S. Patent Application Publication No. 20030162730, published Aug. 28, 2003.

Additional examples of non-dihydrate azithromycin include, but are not limited to, azithromycin monohydrate as disclosed in U.S. Patent Application Publication Nos. 20010047089, published Nov. 29, 2001, and 20020111318, published Aug. 15, 2002, as well as International Application Publication Nos. WO 01/00640, WO 01/49697, WO 02/10181 and WO 02/42315.

Further examples of non-dihydrate azithromycin include, but are not limited to, anhydrous azithromycin as disclosed in U.S. Patent Application Publication No. 20030139583, published Jul. 24, 2003, and U.S. Pat. No. 6,528,492.

Examples of suitable azithromycin salts include, but are not limited to, the azithromycin salts as disclosed in U.S. Pat. No. 4,474,768.

Preferably, at least 70 wt % of the azithromycin in the multiparticulates is crystalline. The degree of azithromycin crystallinity in the multiparticulates can be “substantially crystalline,” meaning that the amount of crystalline azithromycin in the multiparticulates is at least about 80%, “almost completely crystalline,” meaning that the amount of crystalline azithromycin is at least about 90%, or “essentially crystalline,” meaning that the amount of crystalline azithromycin in the multiparticulates is at least 95%.

The crystallinity of azithromycin in the multiparticulates may be determined using Powder X Ray Diffraction (PXRD) analysis. In an exemplary procedure, PXRD analysis may be performed on a Bruker AXS D8 Advance diffractometer. In this analysis, samples of about 500 mg are packed in Lucite sample cups and the sample surface smoothed using a glass microscope slide to provide a consistently smooth sample surface that is level with the top of the sample cup. Samples are spun in the (p plane at a rate of 30 rpm to minimize crystal orientation effects. The X-ray source (S/B KCu_{α}, λ=1.54 Å) is operated at a voltage of 45 kV and a current of 40 mA. Data for each sample are collected over a period of from about 20 to about 60 minutes in continuous detector scan mode at a scan speed of about 12 seconds/step and a step size of 0.02°/step. Diffractograms are collected over the 2θ range of 10° to 16°.

The crystallinity of the test sample is determined by comparison with calibration standards as follows. The calibration standards consist of physical mixtures of 20 wt %/80 wt % azithromycin/carrier, and 80 wt %/20 wt % azithromycin/carrier. Each physical mixture is blended together 15 minutes on a Turbula mixer. Using the instrument software, the area under the diffractogram curve is integrated over the 2θ range of 10° to 16° using a linear baseline. This integration range includes as many azithromycin-specific peaks as possible while excluding carrier-related peaks. In addition, the large azithromycin-specific peak at approximately 10° 2θ is omitted due to the large scan-to-scan variability in its integrated area. A linear calibration curve of percent crystalline azithromycin versus the area under the diffractogram curve is generated from the calibration standards. The crystallinity of the test sample is then determined using these calibration results and the area under the curve for the test sample. Results are reported as a mean percent azithromycin crystallinity (by crystal mass).

Crystalline azithromycin is preferred since it is more chemically and physically stable than the amorphous form. The chemical stability arises from the fact that in crystalline form, azithromycin molecules are locked into a rigid three-dimensional structure that is at a low thermodynamic energy state. Removal of an azithromycin molecule from this structure, for example, to react with a carrier, will therefore take a considerable amount of energy. In addition, crystal forces reduce the mobility of the azithromycin molecules in the crystal structure. The result is that the rate of reaction of azithromycin with acid and ester substituents on a carrier is significantly reduced in crystalline azithromycin when compared to formulations containing amorphous azithromycin.

### Formation of Azithromycin Esters

Azithromycin esters can form either through direct esterification or transesterification of the hydroxyl substituents of azithromycin. By direct esterification is meant that an excipient having a carboxylic acid moiety can react with the hydroxyl substituents of azithromycin to form an azithromycin ester. By transesterification is meant that an excipient having an ester substituent can react with the hydroxyl groups, transferring the carboxylate of the carrier to azithromycin, also resulting in an azithromycin ester. Purposeful synthesis of azithromycin esters has shown that the esters typically form at the hydroxyl group attached to the 2′ carbon (C2′) of the desosamine ring; however esterification at the hydroxyls attached to the 4″ carbon on the cladinose ring (C4″) or the hydroxyls attached to the C6, C11, or C12 carbons on the macrolide ring may also occur in azithromycin formulations. An example of a transesterification reaction of azithromycin with a C₁₆ to C₂₂ fatty acid glyceryl triester is shown below.

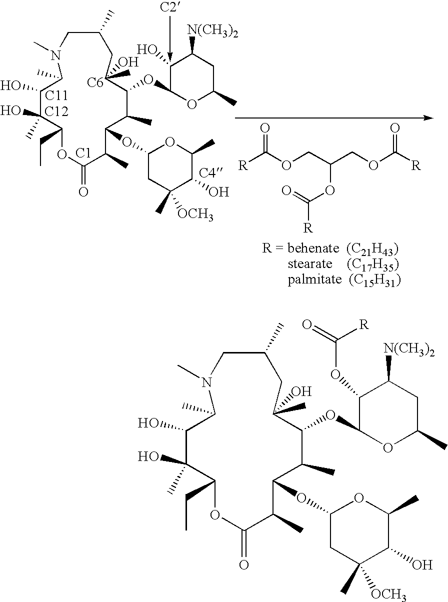

Typically in such reactions, one acid or one ester substituent on the excipient can each react with one molecule of azithromycin, although formation of two or more esters on a single molecule of azithromycin is possible. One convenient way to assess the potential for an excipient to react with azithromycin to form an azithromycin ester is the number of moles or equivalents of acid or ester substituents on the carrier per gram of azithromycin in the composition. For example, if an excipient has 0.13 milliequivalents (meq) of acid or ester substituents per gram of azithromycin in the composition and all of these acid or ester substituents reacted with azithromycin to form mono-substituted azithromycin esters, then 0.13 meq of azithromycin esters would form. Since the molecular weight of azithromycin is 749 g/mole, this means that about 0.1 g of azithromycin would be converted to an azithromycin ester in the composition for every gram of azithromycin initially present in the composition. Thus, the concentration of azithromycin esters in the multiparticulates would be 10 wt %. However, it is unlikely that every acid and ester substituent in a composition will react to form azithromycin esters. As discussed below, the greater the crystallinity of azithromycin in the multiparticulate, the greater can be the concentration of acid and ester substituents on the excipient and still result in a composition with acceptable amounts of azithromycin esters.

The rate of azithromycin ester formation Rₑ in wt %/day for a given excipient at a temperature T(° C.) may be predicted using a zero-order reaction model, according to the following equation:
Rₑ=Cₑₛₜₑᵣₛ÷t (I)
where Cₑₛₜₑᵣₛ is the total concentration of azithromycin esters formed (wt %) and t is time of contact between azithromycin and the excipient in days at temperature T.

One procedure for determining the reaction rate for forming azithromycin esters with the excipient is as follows. The excipient is heated to a constant temperature above its melting point and an equal weight of azithromycin is added to the molten excipient, thereby forming a suspension or solution of azithromycin in the molten excipient. Samples of the molten mixture are then periodically withdrawn and analyzed for formation of azithromycin esters using the procedures described below. The rate of ester formation can then be determined using Equation I above.

Alternatively, the excipient and azithromycin can be blended at a temperature below the melting temperature of the excipient and the blend stored at a convenient temperature, such as 50° C. Samples of the blend can be periodically removed and analyzed for azithromycin esters, as described below. The rate of ester formation can then be determined using Equation I above.

A number of methods well known in the art can be used to determine the concentration of azithromycin esters in multiparticulates. An exemplary method is by high performance liquid chromatography/mass spectrometry (LC/MS) analysis. In this method, the azithromycin and any azithromycin esters are extracted from the multiparticulates using an appropriate solvent, such as methanol or isopropyl alcohol. The extraction solvent may then be filtered with a 0.45 μm nylon syringe filter to remove any particles present in the solvent. The various species present in the extraction solvent can then be separated by high performance liquid chromatography (HPLC) using procedures well known in the art. A mass spectrometer is used to detect species, with the concentrations of azithromycin and azithromycin esters being calculated from the mass-spectrometer peak areas based on either an internal or external azithromycin control. Preferably, if authentic standards of the esters have been synthesized, external references to the azithromycin esters may be used. The azithromycin ester value is then reported as a percentage of the total azithromycin in the sample.

To satisfy a total azithromycin esters content of less than about 10 wt %, the rate of azithromycin ester formation Rₑ in wt %/day should be
Rₑ≦3.6×10⁸·e^{-7070/(T+273)},
wherein T is the temperature in ° C.

To satisfy the preferred total azithromycin esters content of less than about 5 wt %, the rate of total azithromycin esters formation should be
Rₑ≦1.8×10⁸·e^{-7070/(T+273)}.

To satisfy the more preferred total azithromycin esters content of less than about 1 wt %, the rate of total azithromycin esters formation should be
Rₑ≦3.6×10⁷·e^{-7070/(T+273)}.

To satisfy the even more preferred total azithromycin esters content of less than about 0.5 wt %, rate of total azithromycin esters formation should be
Rₑ≦1.8×10⁷·e^{-7070/(T+273)}.

To satisfy the most preferred total azithromycin esters content of less than about 0.1 wt %, the rate of total azithromycin esters formation should be
Rₑ≦3.6×10⁶·e^{-7070/(T+273)}.

A convenient way to assess the potential for azithromycin to react with an excipient to form azithromycin esters is to ascertain the excipients degree of acid/ester substitution. This can be determined by dividing the number of acid and ester substituents on each excipient molecule by the molecular weight of each excipient molecule, yielding the number of acid and ester substituents per gram of each excipient molecule. As many suitable excipients are actually mixtures of several specific molecule types, average values of numbers of substituents and molecular weight may be used in these calculations. The concentration of acid and ester substituents per gram of azithromycin in the composition may then be determined by multiplying this number by the mass of excipient in the composition and dividing by the mass of azithromycin in the composition. For example, glyceryl monostearate,
CH₃(CH₂)₁₆COOCH₂CHOHCH₂OH
has a molecular weight of 358.6 g/mol and one ester substituent per mole. Thus, the ester substituent concentration per gram of excipient is 1 eq ±358.6 g, or 0.0028 eq/g excipient or 2.8 meq/g excipient. If a multiparticulate is formed containing 30 wt % azithromycin and 70 wt % glyceryl monostearate, the ester substituent concentration per gram of azithromycin would be
2.8 meq/g×70/30=6.5 meq/g.
The above calculation can be used to calculate the concentration of acid and ester substituents on any excipient candidate.

However, in most cases, the excipient candidate is not available in pure form, and may constitute a mixture of several primary molecular types as well as small amounts of impurities or degradation products that could be acids or esters. In addition, many excipient candidates are natural products or are derived from natural products that may contain a wide range of compounds, making the above calculations extremely difficult, if not impossible. For these reasons, the inventors have found that the degree of acid/ester substitution on such materials can often most easily be estimated by using the Saponification Number or Saponification Value of the excipient. The Saponification Number is the number of milligrams of potassium hydroxide required to neutralize or hydrolyze any acid or ester substituents present in 1 gram of the material. Measurement of the Saponification Number is a standard way to characterize many commercially available pharmaceutical excipients and the manufacturer often provides an excipient's Saponification Number. The Saponification Number will not only account for acid and ester substituents present on the excipient itself, but also for any such substituents present due to impurities or degradation products in the excipient. Thus, the Saponification Number will often provide a more accurate measure of the degree of acid/ester substitution in the excipient.

One procedure for determining the Saponification Number of a candidate excipient is as follows. A potassium hydroxide solution is prepared by first adding 5 to 10 g of potassium hydroxide to one liter of 95% ethanol and boiling the mixture under a reflux condenser for about an hour. The ethanol is then distilled and cooled to below 15.5° C. While keeping the distilled ethanol below this temperature, 40 g of potassium hydroxide is dissolved in the ethanol, forming the alkaline reagent. A 4 to 5 g sample of the excipient is then added to a flask equipped with a refluxing condenser. A 50-mL sample of the alkaline reagent is then added to the flask and the mixture is boiled under refluxing conditions until saponification is complete, generally, about an hour. The solution is then cooled and 1 mL of phenolphthalein solution (1% in 95% ethanol) is added to the mixture and the mixture titrated with 0.5 N HCl until the pink color just disappears. The Saponification Number in mg of potassium hydroxide per gram of material is then calculated from the following equation:
Saponification Number=[28.05×(B−S)]÷weight of sample
where B is the number of mL of HCl required to titrate a blank sample (a sample containing no excipient) and S is the number of mL of HCl required to titrate the sample. Further details of such a method for determining the Saponification Number of a material is given in Welcher, Standard Methods of Chemical Analysis (1975). The American Society for Testing and Materials (ASTM) also has established several tests for determining the Saponification Number for various materials, such as ASTM D1387-89, D94-00, and D558-95. These methods may also be appropriate for determining the Saponification Number for a potential excipient.

For some excipients, the processing conditions used to form the multiparticulates (e.g., high temperature) may result in a change in the chemical structure of the excipient, possibly leading to the formation of acid and/or ester substituents, e.g., by oxidation. Thus, the Saponification Number of a excipient should be measured after it has been exposed to the processing conditions anticipated for forming the multiparticulates. In this way, potential degradation products from the excipient that may result in the formation of azithromycin esters can be accounted for.

The degree of acid and ester substitution on a excipient can be calculated from the Saponification Number as follows. Dividing the Saponification Number by the molecular weight of potassium hydroxide, 56.11 g/mol, results in the number of millimoles of potassium hydroxide required to neutralize or hydrolyze any acid or ester substituents present in one gram of the excipient. Since one mole of potassium hydroxide will neutralize one equivalent of acid or ester substituents, dividing the Saponification Number by the molecular weight of potassium hydroxide also results in the number of meq of acid or ester substituents present in one gram of excipient.

For example, glyceryl monostearate can be obtained with a Saponification Number of 165, as specified by the manufacturer. Thus, the degree of acid/ester substitution per gram of glyceryl monostearate or its acid/ester concentration is
165÷56.11=2.9 meq/g excipient.
Using the above example of a composition with 30 wt % azithromycin and 70 wt % glyceryl monostearate, the theoretical concentration of esters formed per gram of azithromycin if all of the azithromycin reacted would be
2.9 meq/g×70/30=6.8 meq/g.

When the multiparticulate comprises two or more excipients, the total concentration of acid and ester groups in all excipients should be used to determine the degree of acid/ester substitution per gram of azithromycin in the multiparticulates. For example, if excipient A has a concentration of acid/ester substituents [A] of 3.5 meq/g azithromycin present in the composition and excipient B has an [A] of 0.5 meq/g azithromycin, and both are present in an amount of 50 wt % of the total amount of excipient in the composition, then the mixture of excipients has an effective [A] of (3.5+0.5)÷2, or 2.0 meq/g azithromycin. In this manner some excipients having much higher degrees of acid/ester substitution may be used in the composition.

Excipients and carriers useful in the present invention can be classified into four general categories (1) non-reactive; (2) low reactivity; (3) moderate reactivity; and (4) highly reactive in relation to their tendency to form azithromycin esters. When an extruder is used to form the molten mixture of carrier, optional excipient and drug, the process of the present invention is particularly useful in forming azithromycin multiparticulates using moderately reactive and highly reactive carriers and optional excipients inasmuch as use of the extruder allows use of much more moderate temperatures prior to the atomization step.

Non-reactive carriers and excipients generally have no acid or ester substituents and are free from impurities that contain acids or esters. Generally, non-reactive materials will have an acid/ester concentration of less than 0.0001 meq/g excipient. Non-reactive carriers and excipients are very rare since most materials contain small amounts of impurities. Non-reactive carriers and excipients must therefore be highly purified. In addition, non-reactive carriers and excipients are often hydrocarbons, since the presence of other elements in the carrier or excipient can lead to acid or ester impurities. The rate of formation of azithromycin esters for non-reactive carriers and excipients is essentially zero, with no azithromycin esters forming under the conditions described above for determining the azithromycin reaction rate with an excipient. Examples of non-reactive carriers and excipients include highly purified forms of the following hydrocarbons: synthetic wax, microcrystalline wax, and paraffin wax.

Low reactivity carriers and excipients also do not have acid or ester substituents, but often contain small amounts of impurities or degradation products that contain acid or ester substituents. Generally, low reactivity carriers and excipients have an acid/ester concentration of less than about 0.1 meq/g of excipient. Generally, low reactivity carriers and excipients will have a rate of formation of azithromycin esters of less than about 0.005 wt %/day when measured at 100° C. Examples of low reactivity excipients include long-chain alcohols, such as stearyl alcohol, cetyl alcohol and polyethylene glycol; and ether-substituted cellulosics, such as microcrystalline cellulose, hydroxypropyl cellulose, hydroxypropyl methyl cellulose and ethylcellulose.

Moderate reactivity carriers and excipients often contain acid or ester substituents, but relatively few as compared to the molecular weight of the excipient. Generally, moderate reactivity carriers and excipients have an acid/ester concentration of about 0.1 to about 3.5 meq/g of excipient. Examples include long-chain fatty acid esters, such as glyceryl monooleate, glyceryl monostearate, glyceryl palmitostearate, polyethoxylated castor oil derivatives, glyceryl dibehenate, and mixtures of mono-, di-, and trialkyl glycerides, including mixtures of glyceryl mono-, di-, and tribehenate, glyceryl tristearate, glyceryl tripalmitate and hydrogenated vegetable oils; and waxes, such as carnauba wax and white and yellow beeswax.

Highly reactive carriers and excipients usually have several acid or ester substituents or low molecular weights. Generally, highly reactive carriers and excipients have an acid/ester concentration of more than about 3.5 meq/g of excipient and have a rate of formation of azithromycin esters of more than about 40 wt %/day at 100° C. Examples include carboxylic acids such as stearic acid, benzoic acid, and citric acid. Generally, the acid/ester concentration on highly reactive carriers and excipients is so high that if these carriers or excipients come into direct contact with azithromycin in the formulation, unacceptably high concentrations of azithromycin esters form during processing or storage of the composition. Thus, such highly reactive carriers and excipients are preferably only used in combination with a carrier or excipient with lower reactivity so that the total amount of acid and ester groups on the carriers and excipients used in the multiparticulate is low.

### Carriers

The multiparticulates comprise a pharmaceutically acceptable carrier. By “pharmaceutically acceptable” is meant the carrier must be compatible with the other ingredients of the composition, and not deleterious to the recipient thereof. The carrier functions as a matrix for the multiparticulate or to affect the rate of release of azithromycin from the multiparticulate, or both. Carriers will generally make up about 10 wt % to about 95 wt % of the multiparticulate, preferably about 20 wt % to about 90 wt % of the multiparticulate, and more preferably about 40 wt % to about 70 wt % of the multiparticulates, based on the total mass of the multiparticulate. The carrier is preferably solid at temperatures of about 40° C. The inventors have found that if the carrier is not a solid at 40° C., there can be changes in the physical characteristics of the composition over time, especially when stored at elevated temperatures, such as at 40° C. Thus, it is preferred that the carrier be a solid at a temperature of about 50° C., more preferably about 60° C. For ease of processing, it is also preferred that the carrier be a fluid or liquid (e.g., molten) at a temperature below about 130° C., preferably below about 115° C., and more preferably below about 100° C. In a preferred embodiment, the carrier has a melting point that is less then the melting point of azithromycin. For example, azithromycin dihydrate has a melting point of 113° C. to 115° C. Thus, when azithromycin dihydrate is used in the multiparticulates of the present invention, it is preferred that the carrier have a melting point that is less than about 113° C.

Examples of carriers suitable for use in the multiparticulates of the present invention include waxes, such as synthetic wax, microcrystalline wax, paraffin wax, carnauba wax, and beeswax; glycerides, such as glyceryl monooleate, glyceryl monostearate, glyceryl palmitostearate, polyethoxylated castor oil derivatives, hydrogenated vegetable oils, glyceryl mono-, di- or tribehenates, glyceryl tristearate, glyceryl tripalmitate; long-chain alcohols, such as stearyl alcohol, cetyl alcohol, and polyethylene glycol; and mixtures thereof.

### Excipients

The multiparticulates may optionally include excipients to aid in forming the multiparticulates, to affect the release rate of azithromycin from the multiparticulates, or for other purposes known in the art.

The multiparticulates may optionally include a dissolution enhancer. Dissolution enhancers increase the rate of dissolution of the drug from the multiparticulate. In general, dissolution enhancers are amphiphilic compounds and are generally more hydrophilic than the carrier. Dissolution enhancers will generally make up about 0.1 to about 30 wt % of the total mass of the multiparticulate. Exemplary dissolution enhancers include alcohols such as stearyl alcohol, cetyl alcohol, and polyethylene glycol; surfactants, such as poloxamers (such as poloxamer 188, poloxamer 237, poloxamer 338, and poloxamer 407), docusate salts, polyoxyethylene alkyl ethers, polyoxyethylene castor oil derivatives, polysorbates, polyoxyethylene alkyl esters, sodium lauryl sulfate, and sorbitan monoesters; sugars such as glucose, sucrose, xylitol, sorbitol, and maltitol; salts such as sodium chloride, potassium chloride, lithium chloride, calcium chloride, magnesium chloride, sodium sulfate, potassium sulfate, sodium carbonate, magnesium sulfate, and potassium phosphate; amino acids such as alanine and glycine; and mixtures thereof. Preferably, the dissolution enhancer is at least one surfactant, and most preferably, the dissolution enhancer is at least one poloxamer.

While not wishing to be bound by any particular theory or mechanism, it is believed that dissolution enhancers present in the multiparticulates affect the rate at which the aqueous use environment penetrates the multiparticulate, thus affecting the rate at which azithromycin is released. In addition, such excipients may enhance the azithromycin release rate by aiding in the aqueous dissolution of the carrier itself, often by solubilizing the carrier in micelles. Further details of dissolution enhancers and selection of appropriate excipients for azithromycin multiparticulates are disclosed in commonly assigned U.S. Patent Application Ser. No. 60/527,319 (“Controlled Release Multiparticulates Formed with Dissolution Enhancers,” Attorney Docket No. PC25016), filed Dec. 4, 2003.

Agents that inhibit or delay the release of azithromycin from the multiparticulates can also be included in the multiparticulates. Such dissolution-inhibiting agents are generally hydrophobic. Examples of dissolution-inhibiting agents include: hydrocarbon waxes, such as microcrystalline and paraffin wax; and polyethylene glycols having molecular weights greater than about 20,000 daltons.

Another useful class of excipients that may optionally be included in the multiparticulates include materials that are used to adjust the viscosity of the molten feed used to form the multiparticulates. Such viscosity-adjusting excipients will generally make up 0 to 25 wt % of the multiparticulate, based on the total mass of the multiparticulate. The viscosity of the molten feed is a key variable in obtaining multiparticulates with a narrow particle size distribution. For example, when a spinning-disc atomizer is employed, it is preferred that the viscosity of the molten mixture be at least about 1 cp and less than about 10,000 cp, more preferably at least 50 cp and less than about 1000 cp. If the molten mixture has a viscosity outside these preferred ranges, a viscosity-adjusting excipient can be added to obtain a molten mixture within the preferred viscosity range. Examples of viscosity-reducing excipients include stearyl alcohol, cetyl alcohol, low molecular weight polyethylene glycol (e.g., less than about 1000 daltons), isopropyl alcohol, and water. Examples of viscosity-increasing excipients include microcrystalline wax, paraffin wax, synthetic wax, high molecular weight polyethylene glycols (e.g., greater than about 5000 daltons), ethyl cellulose, hydroxypropyl cellulose, hydroxypropyl methyl cellulose, methyl cellulose, silicon dioxide, microcrystalline cellulose, magnesium silicate, sugars, and salts.

Other excipients may be added to adjust the release characteristics of the multiparticulates or to improve processing and will typically make up 0 to 50 wt % of the multiparticulate, based on the total mass of the multiparticulate. For example, since the solubility of azithromycin in aqueous solution decreases with increasing pH, a base may be included in the composition to decrease the rate at which azithromycin is released in an aqueous use environment. Examples of bases that can be included in the composition include di- and tribasic sodium phosphate, di- and tribasic calcium phosphate, mono-, di-, and triethanolamine, sodium bicarbonate and sodium citrate dihydrate as well as other oxide, hydroxide, phosphate, carbonate, bicarbonate and citrate salts, including hydrated and anhydrous forms known in the art. Still other excipients may be added to reduce the static charge on the multiparticulates. Examples of such anti-static agents include talc and silicon dioxide. Flavorants, colorants, and other excipients may also be added in their usual amounts for their usual purposes.

In one embodiment, the carrier and one or more optional excipients form a solid solution, meaning that the carrier and one or more optional excipients form a single thermodynamically stable phase. In such cases, excipients that are not solid at a temperature of less than about 40° C. can be used, provided the carrier/excipient mixture is solid at a temperature of up to about 40° C. This will depend on the melting point of the excipients used and the relative amount of carrier included in the composition. Generally, the greater the melting point of one excipient, the greater the amount of a low-melting-point excipient that can be added to the composition while still maintaining a carrier in a solid phase at 40° C. or less.

In another embodiment, the carrier and one or more optional excipients do not form a solid solution, meaning that the carrier and one or more optional excipients form two or more thermodynamically stable phases. In such cases, the carrier/excipient mixture may be entirely molten at processing temperatures used to form multiparticulates or one material may be solid while the other(s) are molten, resulting in a suspension of one material in the molten mixture.

When the carrier and one or more optional excipients do not form a solid solution but one is desired, for example, to obtain a specific controlled-release profile, an additional excipient may be included in the composition to produce a solid solution comprising the carrier, the one or more optional excipients, and the additional excipient. For example, it may be desirable to use a carrier comprising microcrystalline wax and a poloxamer to obtain a multiparticulate with the desired release profile. In such cases a solid solution is not formed, in part due to the hydrophobic nature of the microcrystalline wax and the hydrophilic nature of the poloxamer. By including a small amount of a third component, such as stearyl alcohol, in the formulation, a solid solution can be obtained resulting in a multiparticulate with the desired release profile.

In one embodiment, the azithromycin has a low solubility in the molten carrier. This low solubility will limit the formation of amorphous azithromycin during the multiparticulate formation process, resulting in compositions with low concentrations of azithromycin esters. By “solubility in the molten carrier” is meant the mass of azithromycin dissolved in the carrier divided by the total mass of carrier and dissolved azithromycin at the processing conditions at which the molten mixture is formed. Preferably, the solubility of azithromycin in the carrier is less than about 20 wt %, more preferably less than about 10 wt %, and most preferably less than about 5 wt %. The solubility of azithromycin in a molten carrier may be measured by slowly adding crystalline azithromycin to a molten sample of the carrier and determining the point at which azithromycin will no longer dissolve in the molten sample, either visually or through quantitative analytical techniques, such as light scattering. Alternatively, an excess of crystalline azithromycin may be added to a sample of the molten carrier to form a suspension. This suspension may then be filtered or centrifuged to remove any undissolved crystalline azithromycin and the amount of azithromycin dissolved in the liquid phase can be measured using standard quantitative techniques, such as by high performance liquid chromatography (HPLC). When performing these tests, the activity of water in the carrier, atmosphere, or gas to which the azithromycin is exposed should be kept sufficiently high so that the crystal form of the azithromycin does not change during the test, as previously mentioned.

When azithromycin has a high solubility in the carrier at the processing temperature, the dissolved azithromycin is more reactive than crystalline azithromycin. Thus, in such cases, the carrier's concentration of acid/ester substituents should be low so that the azithromycin multiparticulates formed has acceptably low concentrations of azithromycin esters. Preferably, when the solubility of azithromycin in the carrier at the processing temperature is less than about 20 wt % and the remaining azithromycin in the composition is crystalline, the degree of acid/ester substitution on the carrier should be less than about 1.0 meq/g azithromycin in the composition. That is, if the composition contains 1 gram of azithromycin, the total number of equivalents of acid and ester substituents on the carrier should be less than about 1.0 meq. More preferably the degree of acid/ester substitution on the carrier should be less than about 0.2 meq/g azithromycin, even more preferably less than about 0.1 meq/g azithromycin, and most preferably less than about 0.02 meq/g.

The inventors have found that for multiparticulates with an acceptable amount of azithromycin esters, i.e., less than about 10 wt %, there is a trade-off relationship between the concentration of acid and ester substituents on the carrier and the crystallinity of azithromycin in the multiparticulates. Generally speaking, the greater the crystallinity of azithromycin in the multiparticulates, the greater the degree of the carrier's acid/ester substitution may be to obtain multiparticulates with acceptable amounts of azithromycin esters. This relationship may be quantified by the following mathematical expression:
[A]≦0.4/(1−x) (II)
where [A] is the total concentration of acid/ester substitution on the carrier in meq/g azithromycin and is less than or equal to 2 meq/g, and x is the weight fraction of the azithromycin in the composition that is crystalline. When the carrier comprises more than one excipient, the value of [A] refers to the total concentration of acid/ester substitution on all the excipients that make up the carrier, in units of meq/g azithromycin.

For more preferable multiparticulates having less than about 5 wt % azithromycin esters, the azithromycin and carrier will satisfy the following expression:
[A]≦0.2/(1−x). (III)

For even more preferable multiparticulates having less than about 1 wt % azithromycin esters, the azithromycin and carrier will satisfy the following expression:
[A]≦0.04/(1−x). (IV)

For yet more preferable multiparticulates having less than about 0.5 wt % azithromycin esters, the azithromycin and carrier will satisfy the following expression:
[A]≦0.02/(1−x). (V)

For most preferable multiparticulates having less than about 0.1 wt % azithromycin esters, the azithromycin and carrier will satisfy the following expression:
[A]≦0.004/(1−x). (VI)

From the foregoing mathematical expressions (II)-(VI) the trade-off between the carrier's degree of acid/ester substitution and the crystallinity of azithromycin in the composition can be determined. In any case, it is preferred that carriers with acid/ester concentrations of more than 3.5 meq/g azithromycin not be used, since such high degrees of acid/ester substitution will often lead to compositions containing unacceptably high concentrations of azithromycin esters.

In one embodiment, the multiparticulate comprises about 20 to about 75 wt % azithromycin, about 25 to about 80 wt % of a carrier, and about 0.1 to about 30 wt % of a dissolution enhancer based on the total mass of the multiparticulate.

In a more preferred embodiment, the multiparticulate comprises about 35 wt % to about 55 wt % azithromycin; about 40 wt % to about 65 wt % of an excipient selected from waxes, such as synthetic wax, microcrystalline wax, paraffin wax, carnauba wax, and beeswax; glycerides, such as glyceryl monooleate, glyceryl monostearate, glyceryl palmitostearate, polyethoxylated castor oil derivatives, hydrogenated vegetable oils, glyceryl mono-, di- or tribehenates, glyceryl tristearate, glyceryl tripalmitate and mixtures thereof; and about 0.1 wt % to about 15 wt % of a dissolution enhancer selected from surfactants, such as poloxamers, polyoxyethylene alkyl ethers, polyethylene glycol, polysorbates, polyoxyethylene alkyl esters, sodium lauryl sulfate, and sorbitan monoesters; alcohols, such as stearyl alcohol, cetyl alcohol and polyethylene glycol; sugars, such as glucose, sucrose, xylitol, sorbitol and maltitol; salts, such as sodium chloride, potassium chloride, lithium chloride, calcium chloride, magnesium chloride, sodium sulfate, potassium sulfate, sodium carbonate, magnesium sulfate and potassium phosphate; amino acids, such as alanine and glycine; and mixtures thereof.

In another embodiment, the multiparticulates made by the process of the present invention comprise (a) azithromycin; (b) a glyceride carrier having at least one alkylate substituent of 16 or more carbon atoms; and (c) a poloxamer. At least 70 wt % of the drug in the multiparticulate is crystalline. The choice of these particular carrier excipients allows for precise control of the release rate of the azithromycin over a wide range of release rates. Small changes in the relative amounts of the glyceride carrier and the poloxamer result in large changes in the release rate of the drug. This allows the release rate of the drug from the multiparticulate to be precisely controlled by selecting the proper ratio of drug, glyceride carrier and poloxamer. These matrix materials have the further advantage of releasing nearly all of the drug from the multiparticulate. Such multiparticulates are disclosed more fully in commonly assigned U.S. Patent Application Ser. No. 60/527,329 (“Multiparticulate Crystalline Drug Compositions Having Controlled Release Profiles,” Attorney Docket No. PC25020), filed Dec. 3, 2003.

In one aspect, the multiparticulates are in the form of a non-disintegrating matrix. By “non-disintegrating matrix” is meant that at least a portion of the carrier does not dissolve or disintegrate after introduction of the multiparticulate to an aqueous use environment. In such cases, the azithromycin and optionally a portion of the carriers or optional excipients, for example, a dissolution enhancer, are released from the multiparticulate by dissolution. At least a portion of the carrier does not dissolve or disintegrate and is excreted when the use environment is in vivo, or remains suspended in a test solution when the use environment is in vitro. In this aspect, it is preferred that the carrier have a low solubility in the aqueous use environment. Preferably, the solubility of the carrier in the aqueous use environment is less than about 1 mg/mL, more preferably less than about 0.1 mg/mL, and most preferably less than about 0.01 mg/mL. Examples of suitable low-solubility carriers include waxes, such as synthetic wax, microcrystalline wax, paraffin wax, carnauba wax, and beeswax; glycerides, such as glyceryl monooleate, glyceryl monostearate, glyceryl palmitostearate, glyceryl mono-, di- or tribehenates, glyceryl tristearate, glyceryl tripalmitate and mixtures thereof.

### Controlled Release

Multiparticulate compositions made by the process of the present invention are designed for controlled release of azithromycin after introduction to a use environment. By “controlled release” is meant sustained release, delayed release, and sustained release with a lag time. The composition can operate by effecting the release of azithromycin at a rate sufficiently slow to ameliorate side effects. The composition can also release the bulk of the azithromycin in the portion of the GI tract distal to the duodenum. In the following, reference to “azithromycin” in terms of therapeutic amounts or in release rates is to active azithromycin, i.e., the non-salt, non-hydrated macrolide molecule having a molecular weight of 749 g/mol.

In one aspect, the compositions formed by the inventive process release azithromycin according to the release profiles set forth in commonly assigned U.S. Pat. No. 6,068,859.

In another aspect, the compositions formed by the inventive process, following administration of a dosage form containing the composition to a stirred buffered test medium comprising 900 mL of pH 6.0 Na₂HPO₄ buffer at 37° C., releases azithromycin to the test medium at the following rate: (i) from about 15 to about 55 wt %, but no more than 1.1 gA of the azithromycin in the dosage form at 0.25 hour; (ii) from about 30 to about 75 wt %, but no more than 1.5 gA, preferably no more than 1.3 gA of the azithromycin in the dosage form at 0.5 hour; and (iii) greater than about 50 wt % of the azithromycin in the dosage form at 1 hour after administration to the buffered test medium. In addition, dosage forms containing the inventive compositions exhibit an azithromycin release profile for a patient in the fasted state that achieves a maximum azithromycin blood concentration of at least 0.5 μg/mL in at least 2 hours from dosing and an area under the azithromycin blood concentration versus time curve of at least 10 μg·hr/mL within 96 hours of dosing.

The multiparticulates made by the process of the present invention may be mixed or blended with one or more pharmaceutically acceptable materials to form a suitable dosage form. Suitable dosage forms include tablets, capsules, sachets, oral powders for constitution and the like.

The multiparticulates may also be dosed with alkalizing agents to reduce the incidence of side effects. The term “alkalizing agents”, as used herein, means one or more pharmaceutically acceptable excipients that will raise the pH in a constituted suspension or in a patient's stomach after being orally administered to said patient. Alkalizing agents include, for example, antacids as well as other pharmaceutically acceptable (1) organic and inorganic bases, (2) salts of strong organic and inorganic acids, (3) salts of weak organic and inorganic acids, and (4) buffers. Exemplary alkalizing agents include, but are not limited to, aluminum salts such as magnesium aluminum silicate; magnesium salts such as magnesium carbonate, magnesium trisilicate, magnesium aluminum silicate, magnesium stearate; calcium salts such as calcium carbonate; bicarbonates such as calcium bicarbonate and sodium bicarbonate; phosphates such as monobasic calcium phosphate, dibasic calcium phosphate, dibasic sodium phosphate, tribasic sodium phosphate (TSP), dibasic potassium phosphate, tribasic potassium phosphate; metal hydroxides such as aluminum hydroxide, sodium hydroxide and magnesium hydroxide; metal oxides such as magnesium oxide; N-methyl glucamine; arginine and salts thereof; amines such as monoethanolamine, diethanolamine, triethanolamine, and tris(hydroxymethyl)aminomethane (TRIS); and combinations thereof. Preferably, the alkalizing agent is TRIS, magnesium hydroxide, magnesium oxide, dibasic sodium phosphate, TSP, dibasic potassium phosphate, tribasic potassium phosphate or a combination thereof. More preferably, the alkalizing agent is a combination of TSP and magnesium hydroxide. Alkalizing agents are disclosed more fully for azithromycin-containing multiparticulates in commonly assigned U.S. Patent Application Ser. No. 60/527,084 (“Azithromycin Dosage Forms With Reduced Side Effects,” Attorney Docket No. PC25240), filed Dec. 4, 2003.

The multiparticulates made by the process of the present invention may be post-treated to improve the crystallinity of the drug and/or the stability of the multiparticulate. In one embodiment, the multiparticulates comprise azithromycin and at least one carrier, the carrier having a melting point of Tₘ° C.; the multiparticulates are treated after their formation by at least one of (i) heating the multiparticulates to a temperature of at least about 35° C. and less than about (Tₘ° C.−10° C.) and (ii) exposing the multiparticulates to a mobility-enhancing agent. This post-treatment step results in an increase in drug crystallinity in the multiparticulates and typically an improvement in at least one of the chemical stability, physical stability, and dissolution stability of the multiparticulates. Post-treatment processes are disclosed more fully in commonly assigned U.S. Patent Application Ser. No. 60/527,245, (“Multiparticulate Compositions with Improved Stability,” Attorney Docket No. PC11900) filed Dec. 4, 2003.

Without further elaboration, it is believed that one of ordinary skill in the art can, using the foregoing description, utilize the present invention to its fullest extent. Therefore, the following specific embodiments are to be construed as merely illustrative and not restrictive of the scope of the invention. Those of ordinary skill in the art will understand that known variations of the conditions and processes of the following examples can be used.

### Screening Examples 1-3

The tendency of azithromycin to form esters in melts at different temperatures and for different periods of time was studied. A mixture of glyceryl behenates (13 to 21 wt % monobehenate, 40 to 60 wt % dibehenate, and 21 to 35 wt % tribehenate)(COMPRITOL 888 ATO from Gattefossé Corporation of Paramus, New Jersey), was deposited in 2.5 g samples into glass vials and melted in a temperature-controlled oil bath at 100° C. (Example 1), 90° C. (Example 2), and 80° C. (Example 3). To each of these three melts was then added 2.5 g of azithromycin dihydrate, thereby forming a suspension of the azithromycin in the molten COMPRITOL 888 ATO. After stirring the suspension for 15 minutes, a 50 to 100 mg sample of the suspension was removed from each of the molten samples and congealed by allowing the same to cool to room temperature. With stirring of each suspension continuing, additional samples were collected at 30, 60, and 120 minutes following formation of the suspension. All collected samples were stored at −20° C. until analyzed.

Azithromycin esters were identified in each sample by Liquid Chromatography/Mass Spectrometer (LC/MS) Analysis using a Finnegan LCQ Classic mass spectrometer. Samples having a 1.25 mg/mL concentration of azithromycin were prepared by extraction with isopropyl alcohol and sonicated for 15 minutes. The samples were then filtered with a 0.45 μm nylon syringe filter, then analyzed by HPLC using a Hypersil BDS C18 4.6 mm×250 mm (5 μm) HPLC column on a Hewlett Packard HP1100 liquid chromatograph. The mobile phase employed for sample elution was a gradient of isopropyl alcohol and 25 mM ammonium acetate buffer (pH approximately 7) of the following composition: initial conditions of 50/50 (v/v) isopropyl alcohol/ammonium acetate; the isopropyl alcohol percentage was then increased to 100% over 30 minutes and held at 100% for an additional 15 minutes. The flow rate was 0.80 mL/min. The method used a 75 μL injection volume and a 43° C. column temperature.

LC/MS was used for detection with an Atmospheric Pressure Chemical Ionization (APCI) source used in positive-ion mode with selective ion-monitoring. Azithromycin ester formation was calculated from the mass spectrometer peak areas based on an azithromycin control. The azithromycin ester values are reported as percentages of the total azithromycin in the sample. The results of the tests are reported in Table 1, and indicate that the longer the azithromycin was in the molten suspension, and the higher the melt temperature, the greater was the concentration of azithromycin esters.

TABLE 1

| Screening | Melt | Exposure | Ester Concentration |
|---|---|---|---|
| Example | Temperature | Time (min) | (wt %) |
| 1 | 100° C. | 0 | 0.00 |
| | | 15 | 0.13 |
| | | 30 | 0.34 |
| | | 60 | 0.38 |
| | | 120 | 0.92 |
| 2 | 90° C. | 0 | 0.00 |
| | | 15 | 0.09 |
| | | 30 | 0.19 |
| | | 60 | 0.35 |
| | | 120 | 0.49 |
| 3 | 80° C. | 0 | 0.00 |
| | | 15 | 0.05 |
| | | 30 | 0.13 |
| | | 60 | 0.15 |
| | | 120 | 0.38 |

These data were then fitted to Equation I above to describe the rate of azithromycin ester formation Rₑ in wt %/day at the melt temperature used:
Rₑ=Cₑₛₜₑᵣₛ÷t.

The reaction rates calculated from the data in Table 1 are reported in Table 2.

TABLE 2

| Screening | Melt | Rₑ |
|---|---|---|
| Example | Temperature | (wt %/day) |
| 1 | 100° C. | 10.4 |
| 2 | 90° C. | 5.8 |
| 3 | 80° C. | 4.4 |

### Screening Examples 4-25

The tendency of azithromycin to form esters in melts at different temperatures and for different periods of time was studied. Screening Examples 4-25 were prepared like Examples 1-3 except that a variety of different excipients, temperatures, and exposure times were used, all as tabulated in Table 3. The chemical makeup of the various carriers screened is as follows: MYVAPLEX 600 is a glyceryl monostearate; GELUCIRE 50/13 is a mixture of mono-, di- and tri-alkyl glycerides and mono- and di-fatty acid esters of polyethylene glycol; carnauba wax is a complex mixture of esters of acids and hydroxyacids, oxypolyhydric alcohols, hydrocarbons, resinous matter, and water; microcrystalline wax is a petroleum-derived mixture of straight chain and randomly branched saturated alkanes obtained from petroleum; paraffin wax is a purified mixture of solid saturated hydrocarbons; stearyl alcohol is 1-octadecanol; stearic acid is octadecanoic acid; PLURONIC F127 is a block copolymer of ethylene oxide and propylene oxide, referred to as poloxamer 407, and also sold as LUTROL F127 (BASF Corporation of Mt. Olive, N.J.); PEG 8000 is a polyethylene glycol having a molecular weight of 8000 daltons; BRIJ 76 is a polyoxyl 10 stearyl ether; MYRJ 59 is a polyoxyethylene stearate; TWEEN 80 is a polyoxyethylene 20 sorbitan monooleate. Table 3 also reports the concentration of azithromycin esters formed. Table 4 shows the calculated reaction rates.

TABLE 3

| | | Melt | | Esters |
|---|---|---|---|---|
| Screening | | Temperature | Exposure | Formed |
| Example | Excipient | (° C.) | (min) | (wt %) |
| 4 | MYVAPLEX | 100 | 0 | 0 |
| | 600 | | 15 | 0.60 |
| | | | 30 | 1.14 |
| | | | 60 | 1.90 |
| | | | 120 | 3.28 |
| 5 | MYVAPLEX | 90 | 0 | 0 |
| | 600 | | 15 | 0.37 |
| | | | 30 | 0.87 |
| | | | 60 | 1.33 |
| | | | 120 | 1.93 |
| 6 | MYVAPLEX | 80 | 0 | 0 |
| | 600 | | 15 | 0.26 |
| | | | 30 | 0.55 |
| | | | 60 | 0.92 |
| | | | 120 | 1.71 |
| 7 | GELUCIER | 80 | 0 | 0 |
| | 50/13 | | 60 | 0.035 |
| | | | 120 | 0.049 |
| 8 | GELUCIER | 100 | 0 | 0 |
| | 50/13 | | 60 | 0.084 |
| | | | 120 | 0.134 |
| 9 | carnauba wax | 90 | 0 | 0 |
| | | | 60 | 0.012 |
| | | | 120 | 0.015 |
| 10 | carnauba wax | 100 | 0 | 0 |
| | | | 60 | 0.012 |
| | | | 120 | 0.015 |
| 11 | microcrystalline | 100 | 0 | 0 |
| | wax | | 120 | 0.002 |
| 12 | paraffin wax | 100 | 0 | 0 |
| | | | 120 | 0.000 |
| 13 | stearyl alcohol | 80 | 0 | 0 |
| | | | 60 | 0.0001 |
| | | | 120 | 0.0003 |
| 14 | stearyl alcohol | 100 | 0 | 0 |
| | | | 60 | 0.0002 |
| | | | 120 | 0.0001 |
| 15 | stearic acid | 80 | 0 | 0 |
| | | | 60 | 0.704 |
| | | | 120 | 1.718 |
| 16 | stearic acid | 100 | 0 | 0 |
| | | | 60 | 3.038 |
| | | | 120 | 5.614 |
| 17 | PLURONIC | 80 | 0 | 0 |
| | F127 | | 60 | 0.0001 |
| | | | 120 | 0.0000 |
| 18 | PLURONIC | 100 | 0 | 0 |
| | F127 | | 60 | 0.0005 |
| | | | 120 | 0.0001 |
| 19 | PEG 8000 | 100 | 0 | 0 |
| | | | 60 | 0 |
| | | | 120 | 0 |
| 20 | BRIJ 76 | 80 | 0 | 0 |
| | | | 60 | 0.0014 |
| | | | 120 | 0.0015 |
| 21 | BRIJ 76 | 100 | 0 | 0 |
| | | | 60 | 0.0013 |
| | | | 120 | 0.0081 |
| 22 | MYRJ 59 | 80 | 0 | 0 |
| | | | 60 | 0.0017 |
| | | | 120 | 0.0023 |
| 23 | MYRJ 59 | 100 | 0 | 0 |
| | | | 60 | 0.0027 |
| | | | 120 | 0.0042 |
| 24 | TWEEN 80 | 80 | 0 | 0 |
| | | | 60 | 0.0035 |
| | | | 120 | 0.0136 |
| 25 | TWEEN 80 | 100 | 0 | 0 |
| | | | 60 | 0.0193 |
| | | | 120 | 0.0221 |

TABLE 4

| Screening | | Melt Temp. | Rₑ |
|---|---|---|---|
| Example | Excipient | (° C.) | (wt %/day) |
| 4 | MYVAPLEX 600 | 100 | 38.0 |
| 5 | MYVAPLEX 600 | 90 | 22.5 |
| 6 | MYVAPLEX 600 | 80 | 19.9 |
| 7 | GELUCIER 50/13 | 80 | 0.059 |
| 8 | GELUCIER 50/13 | 100 | 1.64 |
| 9 | carnauba wax | 90 | 0.18 |
| 10 | carnauba wax | 100 | 0.23 |
| 11 | microcrystalline wax | 100 | 0 |
| 12 | paraffin wax | 100 | 0 |
| 13 | stearyl alcohol | 80 | 0.0018 |
| 14 | stearyl alcohol | 100 | 0.0047 |
| 15 | stearic acid | 80 | 20.7 |
| 16 | stearic acid | 100 | 67.4 |
| 17 | PLURONIC F127 | 80 | 0.0005 |
| 18 | PLURONIC F127 | 100 | 0.001 |
| 19 | PEG 8000 | 100 | 0 |
| 20 | BRIJ 76 | 80 | 0.018 |
| 21 | BRIJ 76 | 100 | 0.095 |
| 22 | MYRJ 59 | 80 | 0.029 |
| 23 | MYRJ 59 | 100 | 0.051 |
| 24 | TWEEN 80 | 80 | 0.16 |
| 25 | TWEEN 80 | 100 | 0.27 |

The high reaction rates for MYVAPLEX 600 and stearic acid indicate carriers are not suitable candidates.

### Screening Example 26

This example illustrates how the degree of acid/ester substitution can be determined from the Saponification Number for an excipient. The degree of acid/ester substitution [A] for the excipients listed in Table 5 was determined by dividing by 56.11 the Saponification Number for the carrier as listed in Pharmaceutical Excipients 2000.

TABLE 5

| | Saponification | |
|---|---|---|
| Excipients | Number | [A]* |
| hydrogenated castor oil | 176-182 | 3.1-3.2 |
| cetostearyl alcohol | <2 | <0.04 |
| cetyl alcohol | <2 | <0.04 |
| glyceryl monooleate | 160-170 | 2.9-3.0 |
| glyceryl monostearate | 155-165 | 2.8-2.9 |
| glyceryl palmitostearate | 175-195 | 3.1-3.5 |
| Lecithin | 196 | 3.5 |
| polyoxyethylene alkyl ether | <2 | <0.04 |
| polyoxyethylene castor oil derivatives | 40-50 | 0.7-0.9 |
| polyoxyethylene sorbitan fatty acid | 45-55 | 0.8-1.0 |
| esters | | |
| polyoxyethylene stearates | 25-35 | 0.4-0.6 |
| sorbitan monostearate | 147-157 | 2.6-2.8 |
| stearic acid | 200-220 | 3.6-3.9 |
| stearyl alcohol | <2 | <0.04 |
| anionic emulsifying wax | <2 | <0.04 |
| carnauba wax | 78-95 | 1.4-1.7 |
| cetyl esters wax | 109-120 | 1.9-2.1 |
| microcrystalline wax | 0.05-0.1 | 0.001-0.002 |
| nonionic emulsifying wax | <14 | <0.25 |
| white wax | 87-104 | 1.6-1.9 |
| yellow wax | 87-102 | 1.6-1.8 |

*meq/g carrier

### Screening Example 27

This example illustrates how the degree of acid/ester substitution can be determined from the Saponification Number for an excipient. The degree of acid/ester substitution for the excipients listed in Table 6 were determined by dividing by 56.11 the Saponification Number provided by the manufacturer.

TABLE 6

| | Saponification | |
|---|---|---|
| Excipient | Number | [A]* |
| COMPRITOL 888 ATO | 145-165 | 2.6-2.9 |
| GELUCIER 50/13 | 67-81 | 1.2-1.4 |

*meq/g carrier

### Screening Example 28

This example illustrates how the degree of acid/ester substitution can be determined from the structure of the excipient. The degree of acid/ester substitution for the excipients listed in Table 7 was determined by dividing the number of moles of acid and ester substituents on the excipient by its molecular weight. For polymers, the degree of acid/ester substitution was calculated by dividing the average number of moles of acid and ester substituents on the monomer by the monomer's molecular weight.

TABLE 7

| | Molecular | Acid and Ester | |
|---|---|---|---|
| | Weight | Substituents | |
| Excipient | (g/mol) | per mol | [A]* |
| PLURONIC F127 | 10,000 | 0 | 0 |
| paraffin wax | 500 | 0 | 0 |
| PEG 8000 | 8,000 | 0 | 0 |
| Triacetin | 218 | 3 | 14 |

*meq/g carrier

### Screening Example 29

The solubility of azithromycin dihydrate in beeswax was measured using the following procedure. A 5 g sample of beeswax was placed in a glass vial and melted at 65° C. by placing the vial in a hot-water bath. Crystals of azithromycin dihydrate were then slowly added to the molten wax, with stirring. The crystals first added dissolved into the wax. When a total of 0.3 g a azithromycin dihydrate had been added to the molten wax, all of the azithromycin dihydrate dissolved into the wax, whereas when an additional 0.1 gm of azithromycin dihydrate was added, the crystals did not dissolve after stirring for 30 minutes. Thus, the solubility of azithromycin dihydrate in beeswax was determined to be about 6 wt %.

### Screening Examples 30-40

Using the procedure outlined in Screening Example 29, the solubility of azithromycin dihydrate in the excipients listed in Table 8 was determined at the temperatures listed therein. In addition, the solubility of azithromycin dihydrate was determined for mixtures of carriers in the weight ratios reported in Table 8.

TABLE 8

| | | | Azithromycin |
|---|---|---|---|
| Screening | | Temperature | Solubility |
| Example | Excipient | (° C.) | (wt %) |
| 30 | carnauba wax | 95 | 6 |
| 31 | COMPRITOL 888 ATO | 85 | 6 |
| | (glyceryl behenate) | | |
| 32 | paraffin wax | 75 | 5 |
| 33 | MYVAPLEX 600P (glyceryl | 90 | >75 |
| | monostearate) | | |
| 34 | GELUCIRE 50/13 | 90 | 67 |
| 35 | MYRJ 59 (polyoxyethylene | 90 | <1 |
| | stearate) | | |
| 36 | BRIJ 76 (polyoxyethylene | 90 | 1 |
| | alkyl ether) | | |
| 37 | stearyl alcohol | 95 | 60 |
| 38 | 4:1 COMPRITOL 888 | 100 | 25 |
| | ATO:PLURONIC F127 | | |
| 39 | 4:1 carnauba wax:PLURONIC | 90 | 13 |
| | F127 | | |
| 40 | 4:1 COMPRITOL 888 | 85 | 7.5 |
| | ATO:GELUCIRE 51/13 | | |

### Example 1

This example illustrates forming multiparticulates by extruding a molten mixture to an atomizer and congealing the resulting droplets. Multiparticulates comprising 50 wt % azithromycin dihydrate, 45 wt % COMPRITOL 888 ATO, and 5 wt % PLURONIC F127 were prepared using the following melt-congeal procedure. First, 112.5 g of the COMPRITOL, 12.5 g of the PLURONIC F127, and 2 g of water were added to a sealed, jacketed stainless-steel tank equipped with a mechanical mixing paddle. Heating fluid at 97° C. was circulated through the jacket of the tank. After about 40 minutes, the mixture had melted, having a temperature of about 95° C. This mixture was then mixed at 370 rpm for 15 minutes. Next, 125 g of azithromycin dihydrate that had been pre-heated at 95° C. and 100% RH was added to the melt and mixed at a speed of 370 rpm for 5 minutes, resulting in a feed suspension of the azithromycin dihydrate in the molten components.

Using a gear pump, the feed suspension was then pumped at a rate of 250 g/min to the center of a spinning-disk atomizer. The spinning disk atomizer, which was custom made, consists of a bowl-shaped stainless steel disk of 10.1 cm (4 inches) in diameter. The surface of the disk is heated with a thin film heater beneath the disk to about 100° C. That disk is mounted on a motor that drives the disk of up to approximately 10,000 RPM. The entire assembly is enclosed in a plastic bag of approximately 8 feet in diameter to allow congealing and to capture microparticulates formed by the atomizer. Air is introduced from a port underneath the disk to provide cooling of the multiparticulates upon congealing and to inflate the bag to its extended size and shape.

A suitable commercial equivalent, to this spinning disk atomizer, is the FX1 100-mm rotary atomizer manufactured by Niro A/S (Soeborg, Denmark).

The surface of the spinning disk atomizer was maintained at 100° C., and the disk was rotated at 7500 rpm, while forming the azithromycin multiparticulates.

The particles formed by the spinning-disk atomizer were congealed in ambient air and a total of 205 g of multiparticulates collected. The mean particle size was determined to be 170 μm using a Horiba LA-910 particle size analyzer. Samples of the multiparticulates were also evaluated by PXRD, which showed that 83±10% of the azithromycin in the multiparticulates was crystalline dihydrate.

The rate of release of azithromycin from these multiparticulates was determined using the following procedure. A 750 mg sample of the multiparticulates was placed into a USP Type 2 dissoette flask equipped with Teflon-coated paddles rotating at 50 rpm. The flask contained 750 mL of 0.01 N HCl (pH 2) simulated gastric buffer held at 37.0±0.5° C. The multiparticulates were pre-wet with 10 mL of the simulated gastric buffer before being added to the flask. A 3-mL sample of the fluid in the flask was then collected at 5, 15, 30, and 60 minutes following addition of the multiparticulates to the flask. The samples were filtered using a 0.45-μm syringe filter prior to analyzing via HPLC (Hewlett Packard 1100, Waters Symmetry C₈ column, 45:30:25 acetonitrile:methanol:25 mM KH₂PO₄ buffer at 1.0 mL/min, absorbance measured at 210 nm with a diode array spectrophotometer).

The results of this dissolution test are reported in Table 9, and show that a controlled release of azithromycin from the multiparticulate cores was achieved.

TABLE 9

| | Azithromycin |
|---|---|
| Time | Released |
| (min) | (%) |
| 0 | 0 |
| 5 | 7.5 |
| 15 | 24.6 |
| 30 | 44.7 |
| 60 | 73.0 |

Samples of the multiparticulates were analyzed for azithromycin esters by LC/MS as in Screening Examples 1-3. The results of this analysis showed that the concentration of azithromycin esters in the multiparticulates was 0.05 wt %.

### Example 2

Multiparticulates comprising 50 wt % azithromycin dihydrate, 40 wt % COMPRITOL 888 ATO, and 10 wt % PLURONIC F127 were prepared as in Example 1 except that the suspension was stirred for 15 minutes after adding the azithromycin dihydrate to the molten COMPRITOL 888 ATO and PLURONIC F127 and before forming the multiparticulates using the spinning-disk atomizer. The so-formed multiparticulates had a mean particle diameter of about 170 μm. PXRD analysis indicated that 74±1 0% of the azithromycin in the multiparticulates was crystalline dihydrate.

The rate of release of azithromycin from the multiparticulates was determined as in Example 1. The results of these tests are reported in Table 10.

TABLE 10

| | Azithromycin |
|---|---|
| Time | Released |
| (min) | (%) |
| 0 | 0 |
| 5 | 38.3 |
| 15 | 70.8 |
| 30 | 85.9 |
| 60 | 88.9 |

Samples of the multiparticulates were analyzed for azithromycin esters by LC/MS as in Screening Examples 1-3. The results of this analysis showed that the concentration of azithromycin esters in the multiparticulates was 0.33 wt %. Thus, exposing the azithromycin to the molten carriers for a longer period of time resulted in an increase in the amount of azithromycin esters present in the multiparticulates.

### Example 3

Multiparticulates comprising 50 wt % azithromycin dihydrate, 45 wt % carnauba wax, and 5 wt % PLURONIC F127 were prepared using the following melt-congeal procedure. First, 112.5 g of the carnauba wax and 12.5 g of the PLURONIC F127 were melted in a vessel at a temperature of about 93° C. Next, 125 g of azithromycin dihydrate was suspended in this melt and mixed by hand for about 15 minutes, resulting in a feed suspension of the azithromycin dihydrate in the molten components.

Using a gear pump, the feed suspension was then pumped at a rate of 250 g/min to the center of the spinning-disk atomizer of Example 1, rotating at 5000 rpm, the surface of which was maintained at about 98° C. The particles formed by the spinning-disk atomizer were congealed in ambient air and a total of 167 g of multiparticulates collected.

The rate of release of azithromycin from these multiparticulates was determined as in Example 1. The results of this dissolution test are reported in Table 11, and show a controlled release of azithromycin from the multiparticulate cores was achieved.

TABLE 11

| | Azithromycin |
|---|---|
| Time | Released |
| (min) | (%) |
| 0 | 0 |
| 5 | 4 |
| 10 | 7 |
| 15 | 12 |
| 30 | 28 |
| 45 | 40 |
| 60 | 50 |

Samples of the multiparticulates were stored at room temperature for about 190 days and then analyzed for azithromycin esters by LC/MS as in Screening Examples 1-3. The results of this analysis showed that the concentration of azithromycin esters in the multiparticulates was 0.012 wt %.

### Example 4

Multiparticulates comprising 40 wt % azithromycin dihydrate and 60 wt % microcrystalline wax were prepared using the following melt-congeal procedure. First, 150 g of microcrystalline wax and 5 g of water were added to a sealed, jacketed stainless-steel tank equipped with a mechanical mixing paddle. Heating fluid at 97° C. was circulated through the jacket of the tank. After about 40 minutes, the wax had melted, having a temperature of about 94° C. Next, 100 g of azithromycin dihydrate that had been preheated at 95° C. and 100% RH and 2 g of water were added to the melted wax and mixed at a speed of 370 rpm for 75 minutes, resulting in a feed suspension of the azithromycin dihydrate in microcrystalline wax.

Using a gear pump, the feed suspension was then pumped at a rate of 250 cc/min to the center of the spinning-disk atomizer of Example 1, rotating at 7500 rpm, the surface of which was maintained at 100° C. The particles formed by the spinning-disk atomizer were congealed in ambient air. The mean particle size was determined to be 170 μm using a Horiba LA-910 particle-size analyzer. Samples of the multiparticulates were also evaluated by PXRD, which showed that 93±1 0% of the azithromycin in the multiparticulates was crystalline dihydrate.

The rate of release of azithromycin from these multiparticulates was determined as in Example 1. The results of this dissolution test are reported in Table 12, and show that a controlled release of azithromycin from the cores was achieved.

TABLE 12

| | Azithromycin |
|---|---|
| Time | Released |
| (min) | (%) |
| 0 | 0 |
| 15 | 16 |
| 30 | 33 |
| 60 | 46 |

### Example 5

Multiparticulates of the same composition as those in Example 4 were prepared as in Example 4, except that the azithromycin dihydrate was preheated to 100° C. at ambient relative humidity and no additional water was added to the feed tank when the azithromycin dihydrate was mixed with the molten microcrystalline wax. The mean particle size was determined to be 180 μm using a Horiba LA-910 particle-size analyzer. Samples of the multiparticulates were also evaluated by PXRD, which showed that only 67% of the azithromycin in the multiparticulates was crystalline, and dihydrate and non-dihydrate crystalline forms were present in the multiparticulates.

Samples of the multiparticulates were analyzed for azithromycin esters as in Screening Examples 1-3. The results of this analysis showed that the concentration of azithromycin esters in the multiparticulates was less than 0.01 wt %.

### Example 6

Multiparticulates comprising 40 wt % azithromycin dihydrate, 59 wt % microcrystalline wax, and 1 wt % PLURONIC F127 were prepared using the following melt-congeal procedure. First, 200 g of azithromycin dihydrate, 295 g of microcrystalline wax, and 5 g of the PLURONIC F127 were blended in a twin-shell blender for 10 minutes. This blend was then de-lumped in a Fitzpatric L1A mill at 3000 rpm with knives forward using a 0.050″ screen. The blend was then mixed for an additional 10 minutes in a twin-shell blender.

Next, 250 g of this blend was added to a sealed, jacketed stainless-steel tank equipped with a mechanical mixing paddle. Heating fluid at 99° C. was circulated through the jacket of the tank. After about 60 minutes, the blend had melted, and 1 g of water was added to the tank and mixed at 370 rpm. After 15 minutes of mixing, an additional 1 g of water was added to the tank. This was repeated until a total of 4 g of water had been added to the tank.

After a total of 60 minutes of mixing, the feed suspension was pumped at a rate of 250 cc/min using a gear pump to the center of the spinning-disk atomizer of Example 1, rotating at 5000 rpm, the surface of which was maintained at 100° C. The particles formed by the spinning-disk atomizer were congealed in ambient air. The mean particle size was determined to be 250 μm using a Horiba LA-910 particle-size analyzer. Samples of the multiparticulates were also evaluated by PXRD, which showed that 16% of the azithromycin in the multiparticulates was crystalline, and dihydrate and non-dihydrate crystalline forms were present in the multiparticulates.

Samples of the multiparticulates were analyzed for azithromycin esters as in Screening Examples 1-3. The results of this analysis showed that the concentration of azithromycin esters in the multiparticulates was less than 0.005 wt %.

The rate of release of azithromycin from these multiparticulates was determined as in Example 1. The results of this dissolution test are reported in Table 13, and confirm that controlled release of azithromycin from the cores was achieved.

TABLE 13

| | Azithromycin |
|---|---|
| Time | Released |
| (min) | (%) |
| 0 | 0 |
| 15 | 51 |
| 30 | 69 |
| 60 | 83 |

### Example 7

Multiparticulates comprising 40 wt % azithromycin dihydrate, 55 wt % microcrystalline wax, and 5 wt % petrolatum were prepared using the following melt-congeal procedure. First, 137.5 g of microcrystalline wax, 12.5 g of petrolatum, and 2 g of water were added to a sealed, jacketed stainless-steel tank equipped with a mechanical mixing paddle. Heating fluid at 101° C. was circulated through the jacket of the tank. After about 50 minutes, the mixture had melted. Next, 100 g of azithromycin dihydrate that had been pre-heated at 95° C. and 100% RH were added to the melt and mixed at a speed of 370 rpm for 75 minutes, resulting in a feed suspension of the azithromycin dihydrate in microcrystalline wax.

Using a gear pump, the feed suspension was then pumped at a rate of 250 cc/min to the center of the spinning-disk atomizer of Example 1, rotating at 7500 rpm, the surface of which was maintained at 100° C. The particles formed by the spinning-disk atomizer were congealed in ambient air. The mean particle size was determined to be 170 μm using a Horiba LA-910 particle-size analyzer. Samples of the multiparticulates were also evaluated by PXRD, which showed that 85±10% of the azithromycin in the multiparticulates was crystalline dihydrate.

Samples of the multiparticulates were analyzed for azithromycin esters as in Screening Examples 1-3. No azithromycin esters were detected in these multiparticulates.

The rate of release of azithromycin from these multiparticulates was determined as in Example 1. The results of this dissolution test are reported in Table 14, and show that controlled release of azithromycin from the cores was achieved.

TABLE 14

| | Azithromycin |
|---|---|
| Time | Released |
| (min) | (%) |
| 0 | 0 |
| 5 | 10 |
| 15 | 28 |
| 30 | 45 |
| 60 | 55 |

### Example 8

Multiparticulates comprising 38 wt % azithromycin dihydrate, 13 wt % Na₃PO₄, 33 wt % microcrystalline wax, 8 wt % PLURONIC F87, and 8 wt % stearyl alcohol were prepared using the following melt-congeal procedure. First, 166.5 g microcrystalline wax, 62.5 g Na₃PO₄, 41.5 g PLURONIC F87 and 41.5 g stearyl alcohol were heated in a glass beaker in a 95° C. water bath. After about 60 minutes, the mixture had melted. Next, 187.5 g of azithromycin dihydrate was added to the melt and mixed using a spatula for about 15 minutes, resulting in a feed suspension of the azithromycin dihydrate and the Na₃PO₄ in the other components.

Using a gear pump, the feed suspension was then pumped at a rate of 250 cc/min to the center of the spinning-disk atomizer of Example 1, rotating at 7000 rpm, the surface of which was maintained at 100° C. The particles formed by the spinning-disk atomizer were congealed in ambient air. The mean particle size was determined to be 250 μm using a Horiba LA-910 particle-size analyzer. Samples of the multiparticulates were also evaluated by PXRD, which showed that about 89% of the azithromycin in the multiparticulates were crystalline dihydrate.

Samples of the multiparticulates were analyzed for azithromycin esters as in Screening Examples 1-3. No azithromycin esters were detected in these multiparticulates.

The rate of release of azithromycin from these multiparticulates was determined as in Example 1. The results of this dissolution test are reported in Table 15, and show that controlled release of azithromycin from the cores was achieved.

TABLE 15

| | Azithromycin |
|---|---|
| Time | Released |
| (min) | (%) |
| 0 | 0 |
| 5 | 38 |
| 10 | 61 |
| 15 | 78 |
| 30 | 90 |
| 45 | 95 |
| 60 | 97 |

### Example 9

Multiparticulates comprising 45 wt % azithromycin dihydrate, 37 wt % microcrystalline wax, 9 wt % PLURONIC F87, and 9 wt % stearyl alcohol were prepared using the following melt-congeal procedure. First, 370 g microcrystalline wax, 90 g PLURONIC F87 and 90 g stearyl alcohol were heated in a glass beaker in a 93° C. water bath. After about 60 minutes, the mixture had melted. Next, 450 g of azithromycin dihydrate was added to the melt and mixed using a spatula for about 25 minutes, resulting in a feed suspension of the azithromycin dihydrate in the other components.

Using a gear pump, the feed suspension was then pumped at a rate of 250 cc/min to the center of the spinning-disk atomizer of Example 1, rotating at 8000 rpm, the surface of which was maintained at 100° C. The particles formed by the spinning-disk atomizer were congealed in ambient air. The mean particle size was determined to be 190 μm using a Horiba LA-910 particle-size analyzer. Samples of the multiparticulates were also evaluated by PXRD, which showed that about 84% of the azithromycin in the multiparticulates were crystalline dihydrate.

Samples of the multiparticulates were analyzed for azithromycin esters as in Screening Examples 1-3. No azithromycin esters were detected in these multiparticulates.

The rate of release of azithromycin from these multiparticulates was determined as in Example 1. The results of this dissolution test are reported in Table 16, and show that controlled release of azithromycin from the cores was achieved.

TABLE 16

| | Azithromycin |
|---|---|
| Time | Released |
| (min) | (%) |
| 0 | 0 |
| 5 | 54 |
| 10 | 83 |
| 15 | 98 |
| 30 | 96 |
| 45 | 95 |
| 60 | 94 |

### Example 10

Multiparticulates comprising 70 wt % azithromycin dihydrate and 30 wt % stearyl alcohol were prepared using the following melt-congeal procedure.

First, 121 g stearyl alcohol was melted in a glass beaker in a 95° C. water bath.

Next, 282 g of azithromycin dihydrate was added to the melt and mixed using a spatula for about 15 minutes, resulting in a feed suspension of the azithromycin dihydrate in stearyl alcohol.

Using a gear pump, the feed suspension was then pumped at a rate of 250 cc/min to the center of the spinning-disk atomizer of Example 1, rotating at 6700 rpm, the surface of which was maintained at about 95° C. The particles formed by the spinning-disk atomizer were congealed in ambient air. The particle size was determined to be about 229 μm using a Horiba LA-910 particle-size analyzer.

Samples of the multiparticulates were analyzed for azithromycin esters as in Screening Examples 1-3. No azithromycin esters were detected in these multiparticulates.

The rate of release of azithromycin from these multiparticulates was determined as in Example 1. The results of this dissolution test are reported in Table 17, and show that controlled release of azithromycin from the cores was achieved.

TABLE 17

| | Azithromycin |
|---|---|
| Time | Released |
| (min) | (%) |
| 0 | 0 |
| 2.5 | 51 |
| 5.0 | 82 |
| 7.5 | 95 |
| 10.0 | 99 |
| 15.0 | 102 |
| 30.0 | 100 |
| 60.0 | 100 |

### Example 11

Multiparticulates were made comprising 50 wt % azithromycin dihydrate, 40 wt % COMPRITOL 888 ATO, and 10 wt % PLURONIC F127 using the following process. First, 250 g azithromycin dihydrate, 200 g of the COMPRITOL 888 ATO, and 50 g of the PLURONIC F127 were blended in a twinshell blender for 20 minutes. This blend was then de-lumped using a Fitzpatrick L1A mill at 3000 rpm, knives forward using a 0.065-inch screen. The mixture was blended again in a twinshell blender for 20 minutes, forming a preblend feed.

The preblend feed was delivered to a B&P 19-mm twin-screw extruder (MP19-TC with a 25 L/D ratio purchased from B & P Process Equipment and Systems, LLC, Saginaw, Mich.) at a rate of 130 g/min, producing a molten feed suspension of the azithromycin dihydrate in COMPRITOL 888 ATO/PLURONIC F127 at a temperature of about 90° C. The feed suspension was then delivered to the spinning-disk atomizer of Example 1, rotating at 5500 rpm. The maximum residence time of azithromycin dihydrate in the twin-screw extruder was about 60 seconds, and the total time the azithromycin dihydrate was exposed to the molten suspension was less than about 3 minutes. The particles formed by the spinning-disk atomizer were congealed in ambient air and a total of 270 g of multiparticulates were collected.

The so-formed multiparticulates were post-treated as follows. Samples of the multiparticulates were placed in a shallow tray at a depth of about 2 cm. This tray was then placed in a controlled atmosphere oven at 47° C. and 70% RH for 24 hours.

### Examples 12-16

Multiparticulates were made as in Example 11 comprising azithromycin dihydrate, COMPRITOL 888 ATO, and PLURONIC F127 in varying ratios with the variables noted in Table 18.

TABLE 18

| | Formulation | | | | | |
|---|---|---|---|---|---|---|
| | (Azithromycin/ | | | | | |
| | COMPRITOL/ | Feed | Disk | Disk | Batch | Post-treatment |
| Ex. | PLURONIC)* | Rate | Speed | Temp | Size | (° C./% RH; |
| No. | (wt %) | (g/min) | (rpm) | (° C.) | (g) | days) |
| 11 | 50/40/10 | 130 | 5500 | 90 | 500 | 47/70; 1 |
| 12 | 50/45/5 | 140 | 5500 | 90 | 491 | 47/70; 1 |
| 13 | 50/46/4 | 140 | 5500 | 90 | 4968 | 40/75; 5 |
| 14 | 50/47/3** | 180 | 5500 | 86 | 1015 | 40/75; 5 |
| 15 | 50/48/2 | 130 | 5500 | 90 | 500 | 47/70; 1 |
| 16 | 50/50/0 | 130 | 5500 | 90 | 500 | 47/70; 1 |

*COMPRITOL = COMPRITOL 888 ATO; PLURONIC = PLURONIC F127
**3.45 wt % water added to preblend feed.

The azithromycin release rate from the multiparticulates of Examples 11-16 was determined using the following procedure. A sample of the multiparticulates was placed into a USP Type 2 dissoette flask equipped with Teflon-coated paddles rotating at 50 rpm. For Examples 11-13 and 16, 1060 mg of multiparticulates were added to the dissolution medium; for Example 14, 1048 mg was added; for Example 15, 1000 mg was added. The flask contained 1000 mL of 50 mM KH₂PO₄ buffer, pH 6.8, maintained at 37.0±0.5° C. The multiparticulates were pre-wet with 10 mL of the buffer before being added to the flask. A 3-mL sample of the fluid in the flask was then collected at 5, 15, 30, 60, 120, and 180 minutes following addition of the multiparticulates to the flask. The samples were filtered using a 0.45-μm syringe filter prior to analyzing via HPLC (Hewlett Packard 1100, Waters Symmetry C₈ column, 45:30:25 acetonitrile:methanol:25 mM KH₂PO₄ buffer at 1.0 mL/min, absorbance measured at 210 nm with a diode array spectrophotometer). The results of these dissolution tests are reported in Table 19 and show that controlled release of azithromycin was achieved.

TABLE 19

| | | Azithromycin |
|---|---|---|
| Example | Time | Released |
| No. | (min) | (%) |
| 11 | 0 | 0 |
| | 5 | 32 |
| | 15 | 67 |
| | 30 | 90 |
| | 60 | 99 |
| | 120 | 99 |
| | 180 | 100 |
| 12 | 0 | 0 |
| | 15 | 28 |
| | 30 | 46 |
| | 60 | 69 |
| | 120 | 87 |
| | 180 | 90 |
| 13 | 0 | 0 |
| | 15 | 25 |
| | 30 | 42 |
| | 60 | 64 |
| | 120 | 86 |
| | 180 | 93 |
| 14 | 0 | 0 |
| | 15 | 14 |
| | 30 | 27 |
| | 60 | 44 |
| | 120 | 68 |
| | 180 | 81 |
| 15 | 0 | 0 |
| | 5 | 3 |
| | 15 | 11 |
| | 30 | 23 |
| | 60 | 41 |
| | 120 | 66 |
| | 180 | 81 |
| 16 | 0 | 0 |
| | 5 | 4 |
| | 15 | 10 |
| | 30 | 19 |
| | 60 | 32 |
| | 120 | 50 |
| | 180 | 62 |

### Examples 17-19

For Examples 17-19, multiparticulates were made as in Example 11 comprising azithromycin dihydrate and COMPRITOL 888 ATO in varying ratios, with the variables noted in Table 20.

TABLE 20

| | Formulation | | | | | |
|---|---|---|---|---|---|---|
| | (Azithromycin/ | Feed | Disk | Disk | Batch | Post-treatment |
| Ex. | COMPRITOL) | Rate | Speed | Temp | Size | (° C./% RH; |
| No. | (wt %) | (g/min) | (rpm) | (° C.) | (g) | days) |
| 17 | 40/60 | 130 | 5000 | 90 | 500 | 47/70; 1 |
| 18 | 30/70 | 130 | 4750 | 90 | 500 | 47/70; 1 |
| 19 | 20/80 | 130 | 4500 | 90 | 500 | 47/70; 1 |

The azithromycin release rates from the multiparticulates of Examples 17-20 were measured as in Examples 11-16, with the following exceptions. For Example 17, the sample size was 1342 mg; for Example 18, the sample size was 1790 mg; and for Example 19, sample size was 2680 mg. The results of these dissolution tests are reported in Table 21 and show that controlled release of azithromycin was achieved, with the rate of release being dependent on multiparticulate composition.

TABLE 21

| | | Azithromycin |
|---|---|---|
| | Time | Released |
| Example No. | (min) | (%) |
| 17 | 0 | 0 |
| | 5 | 1 |
| | 15 | 6 |
| | 30 | 11 |
| | 60 | 19 |
| | 120 | 31 |
| | 180 | 40 |
| 18 | 0 | 0 |
| | 5 | 2 |
| | 15 | 5 |
| | 30 | 9 |
| | 60 | 15 |
| | 120 | 24 |
| | 180 | 31 |
| 19 | 0 | 0 |
| | 5 | 3 |
| | 15 | 4 |
| | 30 | 7 |
| | 60 | 11 |
| | 120 | 18 |
| | 180 | 23 |

### Example 20

Multiparticulates were made as in Example 11 comprising azithromycin dihydrate, hydrogenated cottonseed oil as a carrier (STEROTEX NF from ABITEC Corp. of Columbus, Ohio), and PLURONIC F127 with the variables noted in Table 22.

TABLE 22

| | Formulation | | | | | |
|---|---|---|---|---|---|---|
| | (azithromycin/ | | | | | |
| | STEROTEX/ | Feed | Disk | Disk | Batch | Post-treatment |
| Ex. | PLURONIC) | Rate | speed | Temp | size | (° C./% RH; |
| No. | (wt %) | (g/min) | (rpm) | (° C.) | (g) | days) |
| 20 | 50/46/4 | 140 | 5500 | 85 | 719 | 40/75; 5 |

The azithromycin release rate from the multiparticulates of Example 20 were measured as in Examples 12-16 with a sample size of 1060 mg. The results of this dissolution test are reported in Table 23 and show controlled release of azithromycin was achieved, with the rate of release being dependent on multiparticulate composition.

TABLE 23

| | | Azithromycin |
|---|---|---|
| Example | Time | Released |
| No. | (min) | (%) |
| 20 | 0 | 0 |
| | 15 | 22 |
| | 30 | 36 |
| | 60 | 52 |
| | 120 | 68 |
| | 180 | 74 |

### Example 21

Multiparticulates were made comprising 50 wt % azithromycin dihydrate, 47 wt % COMPRITOL 888 ATO, and 3 wt % PLURONIC F127. First, 15 kg azithromycin dihydrate, 14.1 kg of the COMPRITOL 888 ATO and 0.9 kg of the PLURONIC F127 were weighed and passed through a Quadro 194S Comil mill in the order listed above. The mill speed was set at 600 rpm. The mill was equipped with a No. 2C-075-H050/60 screen (special round), a No. 2C-1607-049 flat-blade impeller, and a 0.225-inch spacer between the impeller and screen. The mixture was blended using a Servo-Lift 100-L stainless-steel bin blender rotating at 20 rpm, for a total of 500 rotations, forming a preblend feed.

The preblend feed was delivered to a Leistritz 50 mm twin-screw extruder (Model ZSE 50, American Leistritz Extruder Corporation, Somerville, N.J.) at a rate of 25 kg/hr. The extruder was operated in co-rotating mode at about 300 rpm, and interfaced with a melt/spray-congeal (MSC) unit. The extruder had nine segmented barrel zones and an overall extruder length of 36 screw diameters (1.8 m). Water was injected into barrel number 4 at a rate of 8.3 g/min. The extruder's rate of extrusion was set such that it produced a molten feed suspension of the azithromycin dihydrate in the COMPRITOL 888 ATO/PLURONIC F127 at a temperature of about 90° C.

The feed suspension was then delivered to the spinning-disk atomizer of Example 1, maintained at 90° C. and rotating at 7600 rpm. The maximum total time the azithromycin dihydrate was exposed to the molten suspension was less than about 10 minutes. The particles formed by the spinning-disk atomizer were cooled and congealed in the presence of cooling air circulated through the product collection chamber. The mean particle size was determined to be 188 μm using a Horiba LA-910 particle size analyzer. Samples of the multiparticulates were also evaluated by PXRD, which showed that about 99% of the azithromycin in the multiparticulates was in the crystalline dihydrate form.

The multiparticulates of Example 21 were post-treated as follows. Samples of the multiparticulates were placed in sealed barrels. The barrels were then placed in a controlled atmosphere chamber at 40° C. for 3 weeks.

The rate of release of azithromycin from the multiparticulates of Example 21 was determined using the following procedure. Approximately 4 g of the multiparticulates (containing about 2000 mgA of the drug) were placed into a 125 mL bottle containing approximately 21 g of a dosing vehicle consisting of 93 wt % sucrose, 1.7 wt % trisodium phosphate, 1.2 wt % magnesium hydroxide, 0.3 wt % hydroxypropyl cellulose, 0.3 wt % xanthan gum, 0.5 wt % colloidal silicon dioxide, 1.9 wt % titanium dioxide, 0.7 wt % cherry flavoring and 1.1 wt % banana flavoring. Next, 60 mL of purified water was added, and the bottle was shaken for 30 seconds. The contents were added to a USP Type 2 dissoette flask equipped with Teflon-coated paddles rotating at 50 rpm. The flask contained 840 mL of 100 mM Na₂HPO₄ buffer, pH 6.0, held at 37.0±0.5° C. The bottle was rinsed twice with 20 mL of the buffer from the flask, and the rinse was returned to the flask to make up a final volume of 900 mL. A 3-mL sample of the fluid in the flask was then collected at 15, 30, 60, 120, and 180 minutes following addition of the multiparticulates to the flask. The samples were filtered using a 0.45-μm syringe filter prior to analyzing via HPLC (Hewlett Packard 1100, Waters Symmetry C₈ column, 45:30:25 acetonitrile:methanol:25 mM KH₂PO₄ buffer at 1.0 mL/min, absorbance measured at 210 nm with a diode array spectrophotometer). The results of this dissolution test are reported in Table 24, and show that sustained release of the azithromycin was achieved.

TABLE 24

| | | Azithromycin |
|---|---|---|
| Example | Time | Released |
| No. | (min) | (%) |
| 21 | 0 | 0 |
| | 15 | 28 |
| | 30 | 48 |
| | 60 | 74 |
| | 120 | 94 |
| | 180 | 98 |

### Example 21

Multiparticulates were made comprising 50 wt % azithromycin dihydrate, 47 wt % COMPRITOL 888 ATO, and 3 wt % LUTROL F127 using the following procedure. First, 140 kg azithromycin dihydrate was weighed and passed through a Quadro Comil 196S with a mill speed of 900 rpm. The mill was equipped with a No. 2C-075-H050/60 screen (special round, 0.075″), a No. 2F-1607-254 impeller, and a 0.225 inch spacer between the impeller and screen. Next, 8.4 kg of the LUTROL and then 131.6 kg of the COMPRITOL 888 ATO were weighed and passed through a Quadro 194S Comil mill. The mill speed was set at 650 rpm. The mill was equipped with a No. 2C-075-R03751 screen (0.075″), a No. 2C-1601-001 impeller, and a 0.225-inch spacer between the impeller and screen. The mixture was blended using a Gallay 38 cubic foot stainless-steel bin blender rotating at 10 rpm for 40 minutes, for a total of 400 rotations, forming a preblend feed.

The preblend feed was delivered to a Leistritz 50 mm twin-screw extruder (Model ZSE 50, American Leistritz Extruder Corporation, Somerville, N.J.) at a rate of about 20 kg/hr. The extruder was operated in co-rotating mode at about 100 rpm, and interfaced with a melt/spray-congeal unit. The extruder had five segmented barrel zones and an overall extruder length of 20 screw diameters (1.0 m). Water was injected into barrel number 2 at a rate of 6.7 g/min (2 wt %). The extruder's rate of extrusion was adjusted so as to produce a molten feed suspension of the azithromycin dihydrate in the COMPRITOL 888 ATO/LUTROL F127 at a temperature of about 90° C.

The feed suspension was delivered to the spinning-disk atomizer of Example 1, rotating at 6400 rpm. The maximum total time the azithromycin dihydrate was exposed to the molten suspension was less than 10 minutes. The particles formed by the spinning-disk atomizer were cooled and congealed in the presence of cooling air circulated through the product collection chamber. The mean particle size was determined to be about 200 μm using a Malvern particle size analyzer.

The so-formed multiparticulates were post-treated by placing a sample in a sealed barrel that was then placed in a controlled atmosphere chamber at 40° C. for 10 days. Samples of the post-treated multiparticulates were evaluated by PXRD, which showed that about 99% of the azithromycin in the multiparticulates was in the crystalline dihydrate form.

The rate of release of azithromycin from these multiparticulates was determined by placing a sample of the multiparticulates containing about 2000 mgA of azithromycin into a 125-mL bottle, along with 19.36 g sucrose, 352 mg trisodium phosphate, 250 mg magnesium hydroxide, 67 mg hydroxypropyl cellulose, 67 mg xanthan gum, 110 mg colloidal silicon dioxide, 400 mg titanium dioxide, 140 mg cherry flavoring and 230 mg banana flavoring. Next, 60 mL of purified water was added, and the bottle was shaken for 30 seconds. The contents were added to a USP Type 2 dissoette flask equipped with Teflon-coated paddles rotating at 50 rpm. The flask contained 840 mL of a buffered test solution comprising 100 mM Na₂HPO₄ buffer, pH 6.0, maintained at 37.0±0.5° C. The bottle was rinsed twice with 20 mL of the buffer from the flask, and the rinse was returned to the flask to make up a 900 mL final volume. A 3 mL sample of the fluid in the flask was then collected at 15, 30, 60, 120, and 180 minutes following addition of the multiparticulates to the flask. The samples were filtered using a 0.45-μm syringe filter prior to analyzing via HPLC (Hewlett Packard 1100, Waters Symmetry C₈ column, 45:30:25 acetonitrile:methanol:25 mM KH₂PO₄ buffer at 1.0 mL/min, absorbance measured at 210 nm with a diode array spectrophotometer). The results of these dissolution tests are given in Table 25, and show that sustained release of azithromycin was achieved.

TABLE 25

| | | | Azithromycin | Azithromycin |
|---|---|---|---|---|
| | | Time | Released | Released |
| Example | Test Medium | (min) | (mg) | (%) |
| 21 | 100 mM | 0 | 0 | 0 |
| | Na₂HPO₄ | 15 | 720 | 36 |
| | buffer, pH 6.0, | 30 | 1140 | 57 |
| | | 60 | 1620 | 81 |
| | | 120 | 1900 | 95 |
| | | 180 | 1960 | 98 |

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

1. A process for the formation of multiparticulates comprising the steps:
(a) forming a molten mixture comprising azithromycin and a pharmaceutically acceptable carrier;
(b) delivering said molten mixture of step (a) to an atomizing means to form droplets from said mixture; and
(c) congealing said droplets from step (b) to form said multiparticulates.

2. The process of claim 1 wherein said molten mixture is formed in an extruder.

3. The process of claim 2 wherein said multiparticulates contain less than about 1 wt % of azithromycin esters.

4. The process of claim 1 wherein said molten mixture is formed at a processing temperature that is at least 10° C. above the melting point of said carrier.

5. The process of claim 1 wherein said molten mixture comprises a suspension of crystalline azithromycin dihydrate in said carrier.

6. The process of claim 1 wherein said molten mixture is at a temperature of at least about 70° C. and less than about 130° C.

7. The process of claim 1 wherein said molten mixture is molten for at least 5 seconds and for less than about 20 minutes prior to forming said droplets in step (b).

8. The process of claim 2 wherein said multiparticulates contain less than about 0.1 wt % of azithromycin esters.

9. The process of claim 1 wherein said multiparticulates comprise about 20 to about 75 wt % of said azithromycin and about 25 to about 80 wt % of said carrier.

10. The process of claim 9 wherein said carrier is selected from the group consisting of waxes, glycerides and mixtures thereof.

11. The process of claim 10 further comprising a dissolution enhancer, said dissolution enhancer comprising about 0.1 to about 30 wt % of said multiparticulate.

12. The process of claim 1 wherein said multiparticulates comprise about 35 to about 55 wt % of said azithromycin.

13. The process of claim 12 wherein said multiparticulates comprise about 40 to about 65 wt % of said carrier and said carrier is selected from the group consisting of waxes, glycerides and mixtures thereof.

14. The process of claim 13 wherein said carrier is selected from the group consisting of synthetic wax, microcrystalline wax, paraffin wax, Carnauba wax, beeswax, glyceryl monooleate, glyceryl monostearate, glyceryl palmitostearate, polyethoxylated castor oil derivatives, hydrogenated vegetable oils, glyceryl mono-, di- or tribehenates, glyceryl tristearate, glyceryl tripalmitate and mixtures thereof.

15. The process of claim 14 wherein said carrier further comprises about 0.1 to about 15 wt % of a dissolution enhancer.

16. The process of claim 15 wherein said dissolution enhancer is selected from the group consisting of poloxamers, polyoxyethylene alkyl ethers, polyethylene glycol, polysorbates, polyoxyethylene alkyl esters, sodium lauryl sulfate, sorbitan monoesters, stearyl alcohol, cetyl alcohol, polyethylene glycol, glucose, sucrose, xylitol, sorbitol, maltitol, sodium chloride, potassium chloride, lithium chloride, calcium chloride, magnesium chloride, sodium sulfate, potassium sulfate, sodium carbonate, magnesium sulfate, potassium phosphate, alanine, glycine and mixtures thereof.
